(19) United States
(12) Patent Application Publication
(10) Pub. No.: US 2005/0240358 A1
(43) Pub. Date: Oct. 27, 2005

(54) Genome information display system

(75) Inventors: Jun Sasaki, Chuo-ku (JP); Tetsuya Ohtani, Chuo-ku (JP); Kazuhiko Akizuki, Chuo-ku (JP); Makoto Akeo, Moriya-shi (JP)

Correspondence Address: CROWELL & MORING LLP;INTELLECTUAL PROPERTY GROUP, P.O. BOX 14300, WASHINGTON, DC, 20044-4300, US

(73) Assignee: Nittetsu Hitachi Systems Engineering, Inc., Chuo-ku (JP)

(21) Appl. No.: 11/110,890

(22) Filed: Apr. 21, 2005

(30) Foreign Application Priority Data
Apr. 27, 2004 (JP) 2004-131668

(51) Int. Cl. G06F 19/00; G01N 33/48; G01N 33/50

(52) U.S. Cl. 702/20

(57) ABSTRACT

The object of the present invention is to provide a genome information display system that allows the user to quickly conduct operations such as movement of display content on the screen presenting information of a gene or the like of a genome.

A server 10, on receipt of a request for map screen from a terminal 60, transmits, to the terminal 60, a mass of map screen data necessary for preparing a map screen. Therefore, for example, when the user conducts operations such as movement, expansion/contraction of the display extension of an enlarged map on a map screen, the terminal 60, instead of making a renewed access to the server 10 for data communication, quickly prepares an enlarged map presenting the display extension according to the operation, based on the mass of map screen data stored in the memory means of the terminal 60, and displays the enlarged map at a high speed matched with the user's operation.

### BACKGROUND OF THE INVENTION

1. Field of the Invention

The present invention relates to a genome information display system that allows, for example, a user who wants to study a genome to review information about the genome.

2. Description of the Related Art Recently, with progress of the research on genomes, multitude sets of data about the base sequences of genes or the like and their functions have been collected. Such genome data are compiled by public data-storage organizations such as US National Center for Biotechnology Information (NCBI) into databases. Such a public data-storage organization permits access by the public to a database of genome data it manages, and offers a display program so that a researcher can review data from the database on his/her terminal using the program. Namely, a user can review data from a database of genome a server opens to the public by executing functions made available by such a display program. Among such display programs that help the user to review genome data, there has been proposed one that allows the user to view information about the sequence data of genes in a graphic form on a variety of formats (see, for example, Japanese Unexamined Patent Application Publication No. 2001-125929).

Conventional display programs for the review of genome data have been designed on the premise that the user communicates information with the server holding a public database on the basis of an HTTP protocol. Because of this, when using such a display program, genome data from the public database are transmitted to the user being segmented into clusters each cluster containing an amount of data corresponding to one page, and the data are displayed as such on a computer of the user. If the user needs to review the data of a further page, he/she must note the number of that page, gain access to the public database, and request the server to transmit the data cluster corresponding to that page number. Accordingly, before the user has that cluster of data on display, inconveniently he/she must wait a certain time. Thus, there is a demand for the advent of a new system that allows the user to quickly conduct operations such as movement of the display content, on the screen presenting genome data.

The present invention was proposed to meet the above demand, and aims to provide a genome information display system that allows the user to quickly conduct operations such as movement of display content on the screen presenting information of a gene or the like of a genome.

### SUMMARY OF THE INVENTION

To achieve the above object, one aspect of the invention as described in claim 1 is to provide a genome information display system comprising a server which manages plural kinds of map screen data each kind of map screen data including the position data of individual elements constituting a genome, and plural terminals which are connected to the server via a network such that a map screen for the graphical representation of each element is displayed on a display means of each terminal, wherein the server comprises: data set storage means for storing the plural kinds of map screen data; search means which, on receipt of search condition data from each terminal in the quest of data stored within the data set storage means, searches through the data stored within the data set storage means for the required data based on the search condition data; and data management means which transmits the search result data obtained as a result of the search by the search means to the terminal, and on receipt of a request for map screen related to a search result data from the terminal, fetches a mass of map screen data necessary for preparing the requested map screen related to the search result data from the data set storage means to transmit that mass of map screen data to the terminal, and wherein each terminal comprises: first display control means which causes a search condition input screen through which the user can enter search condition data in the quest of data within the data set storage means, and a search result display screen for displaying search result data transmitted from the server to be displayed on the display means; communication control means which transmits search condition data fed via the search condition input screen to the server, and transmits, when the user selects one of the search result data presented on the search result display screen, a request for map screen related to the selected search result data to the server; memory means for temporarily storing a mass of map screen data necessary for the preparation of map screen transmitted by the server; and second display control means which, on receipt of a mass of map screen data necessary for the preparation of map screen related to the search result data selected on the search result display screen from the server, prepares an enlarged map graphically presenting the extension in genome in which each element exists such that the element corresponding to the selected search result data comes roughly at the center of the enlarged map, for each kind of map screen data, based on the mass of map screen data transmitted by the server, and causes the enlarged map to be displayed on a first field of the map screen, further, prepares an overall map for presenting the extension in genome corresponding to the mass of map screen data transmitted by the server in form of a band structure, with an indicating means for indicating, on the overall map, the extension in genome presented on the enlarged map, and causes the overall map with the indicating means to be displayed on a second field of the map screen, and furthermore, when the user moves the indicating means on the overall map by operation of an input means, prepares the enlarged map presenting the extension indicated by the moved indicating means based on the mass of map screen data stored within the memory means, and causes the enlarged map to be displayed on the first field.

Another aspect of the invention described in claim 2 is to provide a genome information display system as described in claim 1 wherein a moving operation means for moving the extension in genome presented on the enlarged map is provided in a third field of the map screen, and when the operation of the moving operation means is conducted, the second display control means prepares the enlarged map where the extension in genome to be displayed is moved at a specified speed as long as the operation is conducted, based on the mass of map screen data stored within the memory means, and causes the enlarged map to be displayed on the first field.

Yet another aspect of the invention described in claim 3 is to provide a genome information display system as described in claim 1 or 2 wherein an expanding/contracting operation means for expanding or contracting the extension in genome presented on the enlarged map is provided in a third field of the map screen, and when the operation of the expanding/contracting operation means is conducted, the second display control means prepares the enlarged map where the extension in genome to be displayed is expanded or contracted at a specified speed as long as the operation is conducted, based on the mass of map screen data stored within the memory means, and causes the enlarged map to be displayed on the first field.

Yet another aspect of the invention described in claim 4 is to provide a genome information display system as described in any one of claims 1 to 3 wherein a jumping operation means for specifying the extension in genome by numeric value and displaying the enlarged map about the specified extension in genome on the first field is provided in a third field of the map screen, and when the operation of the jumping operation means is conducted, the second display control means prepares the enlarged map about the extension in genome specified by numeric value via the jumping operation means based on the mass of map screen data stored within the memory means, and causes the enlarged map to be displayed on the first field.

To achieve the above object, yet another aspect of the invention described in claim 14 is to provide a computer-readable recording medium on which is recorded a program for enabling a computer to execute the functions of a genome information display system as described in any one of claims 1 to 13.

To achieve the above object, yet another aspect of the invention described in claim 15 is to provide a program for enabling a computer to execute the functions of a genome information display system as described in any one of claims 1 to 13.

According to the present invention, on receipt of a request for map screen from a terminal, the server transmits a mass of map screen data necessary for the preparation of map screen to the terminal. Therefore, for example, when the user conducts operations such as movement, expansion/contraction of the display extension of an enlarged map on a map screen, the terminal, instead of making a renewed access to the server for data communication, quickly prepares an enlarged map presenting the display extension according to the operation, based on the mass of map screen data stored in the memory means, and displays the enlarged map at a high speed matched with the user's operation.

### BRIEF DESCRIPTION OF THE DRAWINGS

FIG. 1 is a constitution diagram for showing the outline of a genome information display system representing an embodiment of the invention.

FIG. 2 is a schematic block diagram for showing the organization of the server of the genome information display system.

FIG. 3 is a schematic block diagram for showing the organization of each terminal of the genome information display system.

FIG. 4 shows the contents of data stored in each map screen data storage portion included in the data set storage means of the server.

FIG. 5 shows the contents of keyword data stored in a keyword data storage portion included in the data set storage means of the server.

FIG. 6 shows an exemplary authentication screen.

FIG. 7 shows an exemplary search condition input screen and search result display screen.

FIG. 8 shows an exemplary map screen presented as a single screen framing.

FIG. 9 shows an exemplary map screen presented as a synchronous screen framing.

FIG. 10 shows an exemplary map screen presented as a non-synchronous screen framing.

FIG. 11 shows an exemplary display screen for indicating a base sequence.

FIG. 12 shows an exemplary screen framing script for a single screen framing.

FIG. 13 shows an exemplary screen framing script for a synchronous screen framing.

FIG. 14 shows an exemplary screen framing script for a non-synchronous screen framing.

FIG. 15 shows an exemplary layout script.

FIG. 16 is a flowchart for illustrating the procedures for registering map screen data and keyword data into the data set storage means of a genome information display system representing an embodiment of the invention.

FIG. 17 is a flowchart for illustrating the procedures for displaying a map screen on the display means of each terminal in a genome information display system representing an embodiment of the invention.

### DESCRIPTION OF THE PREFERRED EMBODIMENTS

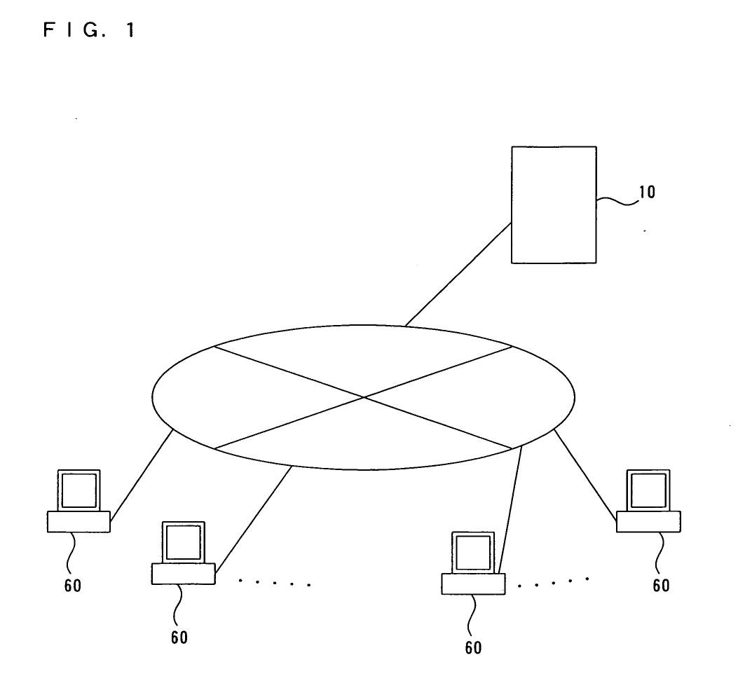

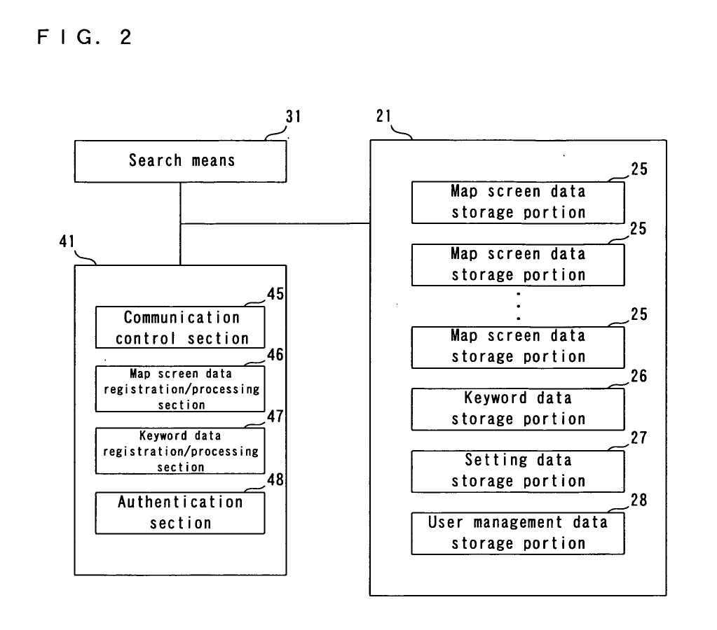

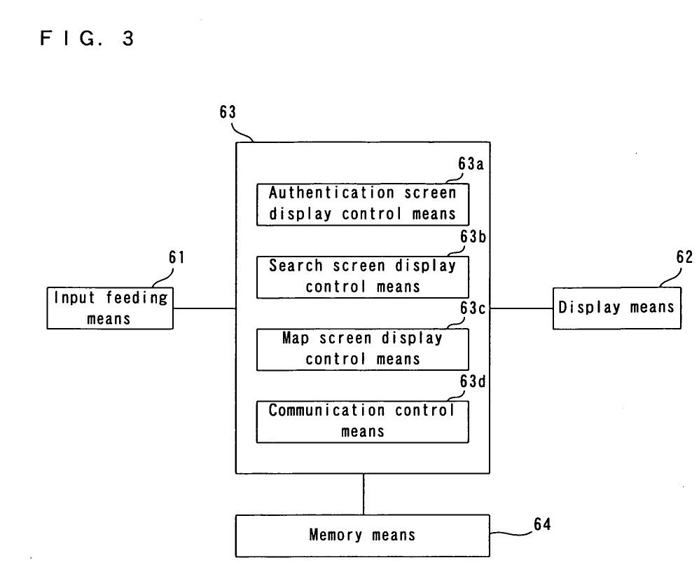

The best mode for carrying out the invention specified in this Application will be described with reference to the attached drawings. FIG. 1 is a constitution diagram for showing the outline of a genome information display system representing an embodiment of the invention. FIG. 2 is a schematic block diagram for showing the organization of the server of the genome information display system. FIG. 3 is a schematic block diagram for showing the organization of each terminal of the genome information display system.

A genome information display system representing an embodiment of the invention comprises a server 10 and a plurality of terminals 60, 60, . . . as shown in FIG. 1. The server 10 and the plurality of terminals 60, 60, . . . are connected to each other via a network. Data can be communicated between the server 10 and the plurality of terminals 60, 60, . . . via the network. The network may include, for example, Internet.

The server 10 manages plural kinds of map screen data including the position data of individual elements such as genes constituting a genome. The server 10 is installed, for example, in a company that utilizes the system or in a company to which the management of the system is assigned. Each terminal 60 receives a specific mass of map screen data from the server 10, and prepares a map screen for the graphical representation of the data of an element based on the received mass of map screen data, and presents the map screen on its display means. The terminal 60 is installed in a research institute or a pharmaceutical company that is engaged with the study of genetics.

The server 10 comprises a data set storage means 21, search means 31, and data management means 41 as shown in FIG. 2. The data set storage means 21 stores plural kinds of map screen data, keyword data, setting data, etc. The configuration of the data set storage means 21 will be detailed later. The search means 31, on receipt of search condition data from a terminal 60 for the search of data stored within the data set storage means 21, searches through the data stored within the data set storage means 21 for the requested data based on the search condition data.

The data management means 41 is for managing data stored within the data set storage means 21. The data management means 41 comprises a communication control section 45, map screen data registration/processing section 46, keyword data registration/processing section 47, and authentication section 48 as shown in FIG. 2. The communication control section 45 transmits search result data obtained by the search means 31 as a result of search to the related terminal 60. In addition, the section 45, on receipt of a request for map screen related to a search result data from a terminal 60, obtains a mass of map screen data necessary for preparing the map screen related to the search result data from the data set storage means, to transmit the data to the terminal 60. The communication control section 45, on receipt of a first access from a terminal 60, transmits a specified display program to the terminal 60. The display program is stored in the data set storage means 21.

The map screen data registration/processing section 46, converts public genome data and/or genome data possessed by a personal user to a specified format, and registers the converted data as a kind of map screen data into the data set storage means 21. The keyword data registration/processing section 47 extracts the names of elements contained in the map screen data as keywords, prepares keyword data using the extracted keywords, and registers the keyword data into the data set storage means 21. The authentication section 48, when receiving a user ID and password from a terminal 60, conducts procedures necessary for the authentication of the user based on the user ID and password provided by the terminal 60.

Next, the configuration of the data set storage means 21 will be described. The data set storage means 21 comprises a plurality of map screen data storage portions 25, 25, . . . , keyword data storage portion 26, setting data storage portion 27, and user management data storage portion 28 as shown in FIG. 2. The plural map screen data storage portions 25, 25, . . . store plural kinds of map screen data in respective map screen data storage portions. Each kind of map screen data includes the position data of individual elements constituting a genome, and is prepared by the map screen data registration/processing section 46 on the basis of public genome data, for example. Suitable public genome data may include, for example, RefSeq Genes, GenScan Gene Predictions, Clone Coverage/Fragment Position, etc. RefSeq Genes is the sequence data of human genome genes constructed by UCSC based on mRNA sequences that are managed in accordance with the Reference Sequence project of NCBI (National Center for Biotechnology Information). GenScan Predictions is the prediction data of genes obtained by applying a gene prediction program (tool) called GenScan to Clone Coverage. Clone Coverage/Fragment Position is the sequence data of genes contained in chromosome fragments each of which is sufficiently small to be treatable in experiment. Both GenScan Predictions and Clone Coverage/Fragment Position were constructed by UCSC. According to this invention, a kind of map screen data prepared from RefSeq Genes, another kind of map screen data prepared from GenScan Gene Predictions, and yet another kind of map screen data prepared from Clone Coverage/Fragment Position are stored in separate map screen data storage portions 25. Because of this arrangement, the data management means 41, when informed, for example, of the title of map screen data, can identify the map screen data storage portion 25 corresponding to the notified title, and gain access to the map screen data having the notified title. Since a kind of map screen data is prepared from a genome data in this embodiment, the kind of map screen data is given a title after the title of the genome data from which it is prepared. Thus, in the following, a kind of map screen data prepared from RefSeq Genes will be called “RefSeq”, that prepared from GenScan Gene Predictions “GenScan”, and that from Clone Coverage/Fragment Position “Coverage”.

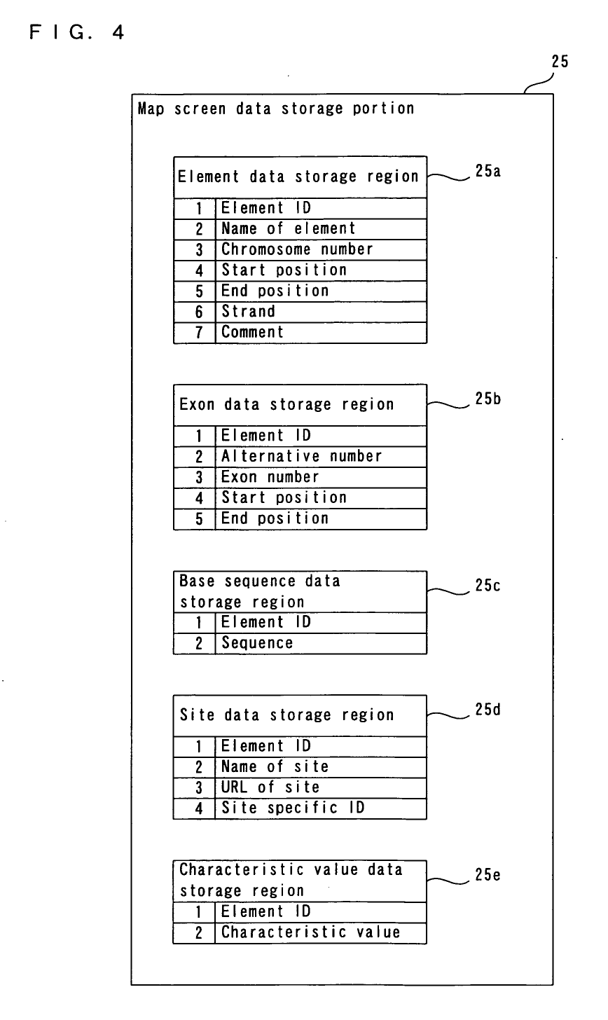

FIG. 4 shows the contents of data stored in each map screen data storage portion 25. The map screen data storage portion 25 comprises an element data storage region 25a, exon data storage region 25b, base sequence data storage region 25c, site data storage region 25d, and characteristic value data storage region 25e as shown in FIG. 4.

The element data storage region 25a stores the data of individual elements in a tabular form. The term “element” as used herein refers to the unit of base sequences that is sufficiently significant to be presented on map screen. The element may include, for example, genes, clones, contigs, etc. In contrast, the exon is not an element. Which element should be used for map screen may be determined as appropriate for each kind of map screen data. For example, genes are taken as elements in RefSeq, while clones are taken as elements in Coverage. The element data includes the “element ID”, “name of element”, “chromosome number”, “start position”, “end position”, “strand”, and “comment” as shown in FIG. 4. The “element ID” refers to identification information attached to the element. The same element ID may be used in duplicate, provided that the two element data having the same element ID belong to different kinds of map screen data. The “name of element” is the name of the element. Usually, the name of an element is the same name as used in the original genome data. Particularly, if the element is a contig, for example, the name of the code attached to the contig may be used as the name of the element.

The “chromosome number”, “start position”, and “end position” represent the position data of the element. The “chromosome number” indicates the number of the chromosome that contains the element. If the genome does not include any chromosome, naturally the element data will not include the data of chromosome number. The “start position” indicates the start position of the extension in genome of the element. The “end position” indicates the end position of the extension in genome of the element. If the genome includes chromosomes, the start and end positions of a given element are determined with respect to the chromosome on which the element resides. To put it more concretely, in this embodiment, for each chromosome, its constitutive base sequences are numbered sequentially so that the position of any given base therein can be identified by a unique number attached thereto. The start and end positions of the element are identified by the position of the base. Therefore, if the chromosome number, and start and end positions of an element are given, the extension in genome of the element is uniquely determined. Description will be given below on the assumption that the genome includes chromosomes.

The DNA has a double helical structure consisting of two strands twisted around each other. The two strands include base sequences complementary to each other. The base sequence data of a gene may be read in two different ways: one base sequence may be read in a 5′-end to 3′-end direction for one strand, and the other base sequence may be read in a 5′-end to 3′-end direction for the other strand. The base sequence data may have + or − attached thereto depending on its direction with respect to the orientation of the chromosome on which the gene resides: the sequence receives positive sign (+) when it extends in a short to long arm direction of the chromosome, while it receives negative sign (−) when it extends in the opposite direction. The symbol information for identifying the direction of a sequence with respect to the orientation of chromosome is the “strand”. The “comment” is the text information about the element, which is included in the original genome data.

The exon data storage region 25b stores the data of exons included in individual elements in a tabular form. The exon data comprises the “element ID”, “alternative number”, “exon number”, “start position”, “end position” as shown in FIG. 4. The “element ID” refers to identification information attached to the element to which an exon under consideration belongs. The “alternative number” is a number for noting alternative splicing. The “exon number” is a number attached to the exon. Based on the exon number, the exon can be uniquely identified among other exons contained in the same element. The “start position” refers to the start position of the extension in the genome of the exon while the “end position” refers to the end position of the extension in the genome of the exon. If the genome includes chromosomes, the start and end positions is determined with respect to the chromosome on which the exon resides.

The base sequence data storage region 25c stores the base sequence data of individual elements in a tabular form. The base sequence data of each element comprises the “element ID” and “sequence” as shown in FIG. 4. The “element ID” refers to identification information attached to the element. The “sequence” is data representing the base sequence of the element, that is, sequence data comprising A (adenine), G (guanine), C (cytosine) and T (thymine).

The site data storage region 25d stores the data about the site(s) providing information about individual elements in a tabular form. The site may include, for example, the site responsible for the management of an original genome data, and the site responsible for the management of the data about the element or data related with the element is stored, etc. The site data includes the “element ID”, “name of site”, “URL of site” and “site specific ID” as shown in FIG. 4. The “element ID” refers to identification information attached to the element related with a site under consideration. The “name of site” is the name of the site. The “URL of site” is the URL of the site. The “site specific ID” is the specific ID provided by the site. For example, when accessing the site that manages the original genome data, the site attaches the unique ID to the data managed by the site, and a user may fail to view a desired data without inputting the unique ID of the data. Such ID attached to the data by the site is the site specific ID. The site specific ID as well as the URL of the site is stored in the data set storage means 21. Thus, the terminal 60 can readily gain access to a site where a desired data resides by utilizing the site data stored in said means, certainly obtain a desired data, and present the data on its display means.

The characteristic value data storage region 25e stores the characteristic value data of individual elements in a tabular form. The characteristic value data includes the “element ID” and “characteristic value” as shown in FIG. 4. The “element ID” is identification information attached to an element under consideration. The “characteristic value” is a characteristic value(s) attached to the element in the original genome data. The characteristic value may include, for example, “score”, “expected value”, “similarity”, etc., obtained as a result of applying a homology determination program (tool) such as Blast to the element.

As described above, all of the element data, exon data, base sequence data, site data and characteristic value data includes element IDs. Thus, the data management means 41, when informed of the title of map screen data and an element ID, can identify the element data, exon data, base sequence data, site data, and characteristic value data of the element to which the element ID is attached, in the map screen data.

As described above, the map screen data includes element data, exon data, base sequence data, site data and characteristic value data. Of them, only the element data and exon data including position data are directly connected with map screen. In contrast, the base sequence data, site data and characteristic value data do not have direct connection with map screen. However, the latter are included in the map screen data. This is because this embodiment is configured so that the user can get many kinds of display, preparation of which needs the latter data, by operating via a menu on map screen. Some map screen data may not include any one or all of exon data, site data and characteristic value data according to the original genome data. However, the map screen data have to include at least the element data.

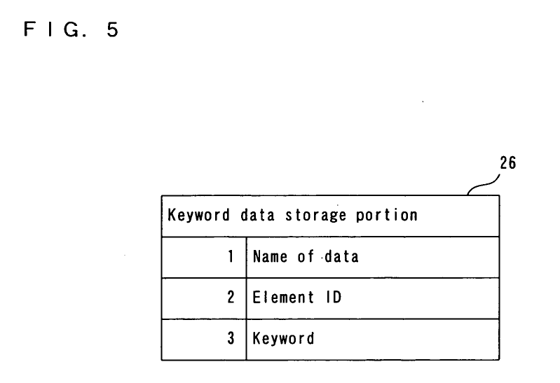

FIG. 5 shows the contents of keyword data stored in a keyword data storage portion 26. The keyword data storage portion 26 stores keyword data in a tabular form. The keyword data is prepared by the keyword data registration/processing section 47. The keyword data includes “name of data”, “element ID” and “keyword” as shown in FIG. 5. The “keyword” is the name of the element. Namely, the keyword is extracted by the keyword data registration/processing section 47 from a kind of map screen data including the data of the element. The “name of data” is the title of the map screen data from which the keyword is extracted. The “element ID” is identification information of the element corresponding to the keyword. Keyword data is connected with the element data through the name of data and element ID.

According to this embodiment, keywords are extracted from each kind of map screen data, and keyword data including keywords extracted as above are stored in the keyword data storage portion 26. These keyword data are the objects through which the search means 31 can search. Specifically, the search means 31, on receipt of search condition data from a terminal 60, gains access first to the keyword data storage portion 26, and locates a keyword corresponding to the search condition data. Then, the search means 31, based on the name of data and element ID corresponding to the keyword, obtains the element data connected with the keyword. The element data thus obtained constitutes search result data. It is also possible for the search means 31 to directly search across element data stored in a plurality of map screen data storage portions 25, 25, . . . However, this, unless otherwise treated, would take a long time. To circumvent the inconvenience, in this embodiment, the keyword data storage portion 26 is provided such that the search means 31 can first search through keyword data stored in the keyword data storage portion 26, which enables the rapid search processing.

The user management data storage portion 28 stores the user IDs and passwords of individual users. The authentication section 48, on receipt of an authentication request together with a user ID and password from a terminal 60, checks whether the user ID and password transmitted correspond with any user ID and password stored in the user management data storage portion 28. Finding that they rightly correspond with certain user ID and password stored in said portion 28, the authentication section 48 authenticates the user, and the communication control section 45 transmits an authentication OK flag to the terminal 60. Once authentication procedures as described above have completed, the terminal 60 is ready to communicate data with the server 10.

The setting data storage portion 27 stores setting data utilized in preparing the map screen. The setting data includes two kinds of data, i.e., screen framing script and layout script. The screen framing script is for setting the framing of map screen. According to this embodiment, the user can choose, for the screen framing of map screen, any one of following three options: single screen framing, synchronous screen framing and non-synchronous screen framing. In correspondence with the three kinds of screen framing, there are three kinds of screen framing scripts. The details of screen framing will be described later. The layout script is for setting the layout of map screen. By way of the layout script, it is possible to specify the title(s), number and order of map screen data displayed on map screen. The screen framing script and layout script are usually prepared in advance by the manager at the server 10.

The setting data are written on a data format called XML. According to XML as in HTML, tags are used, and data are written in a format having a hierarchical structure. Of course, the content of the screen framing or layout of map screen may be specified in a program used for the preparation of a map screen. However, this technique is not practical: if the screen framing or layout of a map screen should be altered, it will be necessary to rewrite the program. Actually, the screen framing and layout of a map screen may often be altered in response to the requests from users. To meet this situation, according to this embodiment, the setting data for setting the screen framing and layout of map screen are separated from the program. Thus, in this embodiment, it is readily possible to alter the screen framing and layout of map screen by modifying only the setting data while leaving the program untouched. The screen framing script and layout script are transmitted to the terminal 60 together with a mass of map screen data necessary for preparing a map screen.

Next, the configuration of each terminal 60 will be described. As shown in FIG. 3, the terminal 60 comprises an input feeding means 61 such as a keyboard, mouse, etc., display means 62 such as a liquid crystal display (LCD), etc., control means 63, and memory means 64. The memory means 64 is for temporarily storing a specific mass of map screen data necessary for preparing a map screen, screen framing script and layout script that have been transmitted by the server 10.

The control means 63 is for generally controlling the entire components constituting the terminal 60. The control means 63 comprises authentication screen display control means 63a, search screen display control means (first display control means) 63b, map screen display control means (second display control means) 63c, and communication control means 63d.

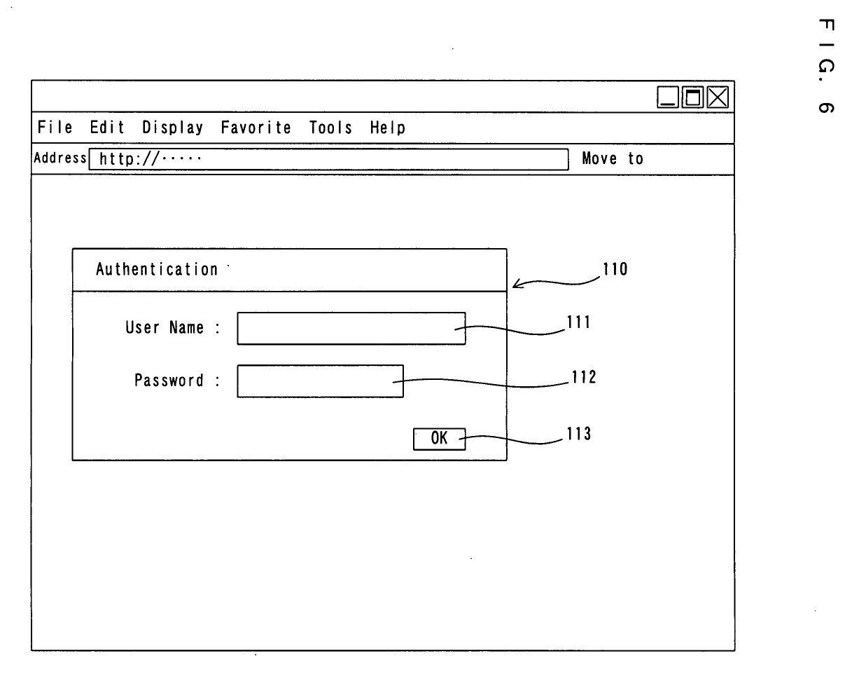

The authentication screen display control means 63a is for displaying a specified authentication screen on the display means 62. In this embodiment, the authentication screen is displayed being embedded in the screen of a Web browser. FIG. 6 shows an exemplary authentication screen. The authentication screen 110 includes an input portion 111 labeled “User Name” into which the user's ID should be entered, and another input portion 112 into which the user's password should be entered. When the user enters his/her user ID and password into the respective portions, and clicks “OK” button 113, the communication control means 63d transmits the user ID and password entered by the user together with an authentication request to the server 10.

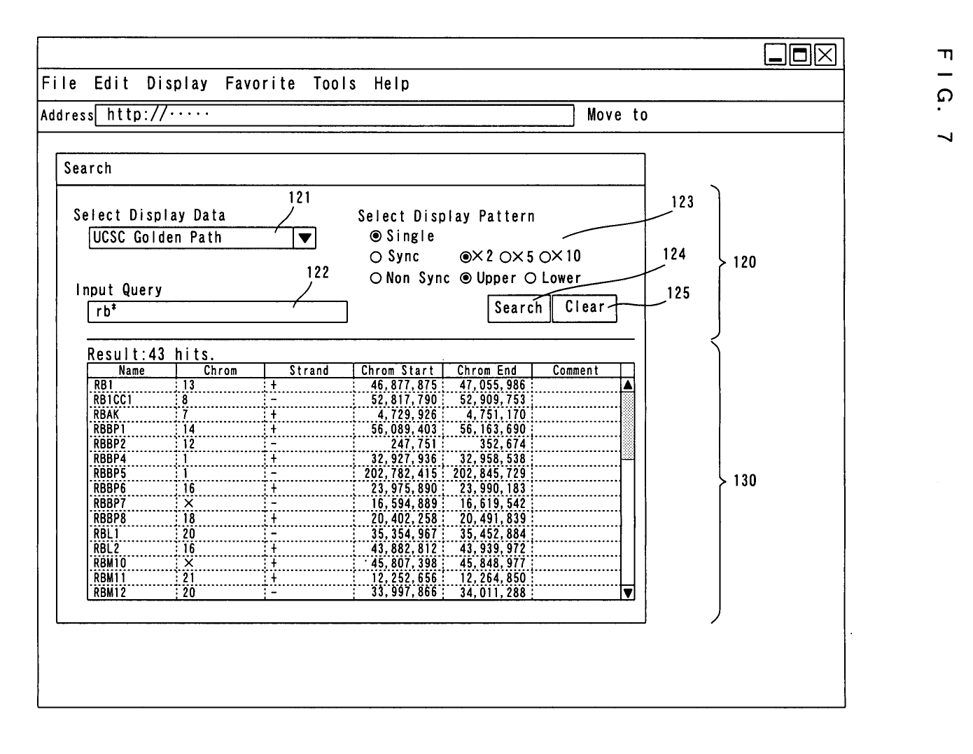

The search screen display control means 63b is for instructing, on receipt of an authentication OK flag from the server 10, the display means 62 to display a search condition input screen so that the user can enter search condition data necessary for the search through data stored in the data set storage means 21 for a desired of data, and a search result display screen for presenting the search result data transmitted from the server 10. Both the search condition input screen and search result display screen are displayed being embedded in the screen of a Web browser. FIG. 7 shows an exemplary search condition input screen and search result display screen. In FIG. 7, the upper half represents a search condition input screen 120 while the lower half a search result display screen 130. The search condition input screen 120 and search result display screen 130 may be called hereinafter collectively a search screen.

As shown in FIG. 7, the search condition input screen 120 includes a map screen data selection portion 121 labeled “Select Display Data”, a search condition input portion 122 labeled “Input Query”, a screen framing selection portion 123 labeled “Select Display Pattern”, and buttons 124 and 125 labeled “Search” and “Clear”, respectively. The map screen data selection portion 121 is for selecting map screen data to be presented on map screen. The map screen data selected via the map screen data selection portion 121 will be map screen data to be searched by the search means 31. In fact, the combo box of map screen data selection portion 121 contains a list of the titles of layout scripts, and when the user selects any desired one from the list, the title selected by the user will be fed into the map screen data selection portion 121. Since each layout script contains the titles of related map screen data to be presented on map screen, this embodiment is configured such that the user can select his/her desired map screen data to be presented on map screen by selecting the title of layout script carrying the titles of those desired map screen data. Accordingly, the map screen data selection portion 121 may be comparable to a layout script selection means for selecting a layout script of the invention. In the particular example shown in FIG. 7, the title of a layout script “UCSC Golden Path” appears in the map screen data selection portion 121. The layout script whose title is “UCSC Golden Path” includes RefSeq, GenScan and Coverage as the titles of map screen data.

The search condition input portion 122 is for entering the name of an element to be searched in the form of a string of characters. The character may include a wild card(s) (*). In the particular example shown in FIG. 7, “rb*” is entered in the search condition input portion 122. Here a chromosome number may be entered. For example, if “chr13” is entered, search will be made for the entire elements contained in chromosome No. 13. The data entered into the map screen data selection portion 121 and the data entered into the search condition input portion 122 constitute search condition data. Incidentally, a button 125 labeled “Clear” is for deleting data that has been entered in the search condition input portion 122.

The screen framing selection portion 123 is for selecting a desired screen framing for map screen. The screen framing selection portion 123 makes available three options, that is, “Single”, “Sync” and “Non Sync.” If the user selects “Single”, a single screen framing will be selected. If the user selects “Sync”, a synchronous screen framing will be selected. If the user selects “Non Sync”, a non-synchronous screen framing will be selected. The map screen display control means 63c prepares, at the time of the display of map screen, the map screen of screen framing in accordance with the selection fed via the screen framing selection portion 123. The content selected via the screen framing selection portion 123 is not search condition data, but the user selects a desired screen framing for map screen at this stage for the convenience of the user's operation.

When the user enters search condition data and clicks “Search” button 124, the communication control means 63d transmits the search condition data together with a search request to the server 10. When the search means 31 of the server 10 receives, from the terminal 60, search condition data including the title of a layout script fed via the map screen data selection portion 121, it obtains, from the data set storage means 21, a layout script corresponding to the title, and determines the kind of map screen data which is the object of the search based on the content of the thus obtained layout script.

The search result display screen 130 gives a list of search result data that have been transmitted by the server 10. The search result data are presented in the items of “Name”, “Chrom”, “Strand”, “Chrom Start”, “Chrom End” and “Comment.” The server 10 retrieves element data for the element corresponding to the search condition data, and transmits the data as search result data to the terminal 60. Because of this, the items attached to the search result display screen 130 are essentially the same with the contents of the element data. Namely, in the item “Name”, the name of the element corresponding to the search condition data is presented. In the item “Chrom”, the number of the chromosome that includes the element is presented. In the item “Strand”, the strand of the element data that includes the element is presented. In the item “Chrom Start”, the start position of the element in the chromosome is presented. In the item “Chrom End”, the end position of the element in the chromosome is presented. In the item “Comment”, the specified comment is presented. If the user selects one search result data in the list of search result data presented in the search result display screen 130, the communication control means 63d transmits, to the server 10, a request for map screen related to the selected search result data together with the chromosome number included in the selected search result data, the title of the layout script fed via the map screen data selection portion 121, and the content of the screen framing selected via the screen framing selection portion 123.

The map screen display control means 63c, on the basis of a mass of map screen data necessary for preparing the map screen from the server 10 together with the specified screen framing script and layout script, prepares the map screen as specified, and presents it on the display means 42. The map screen is distinct from the screen of a Web browser.

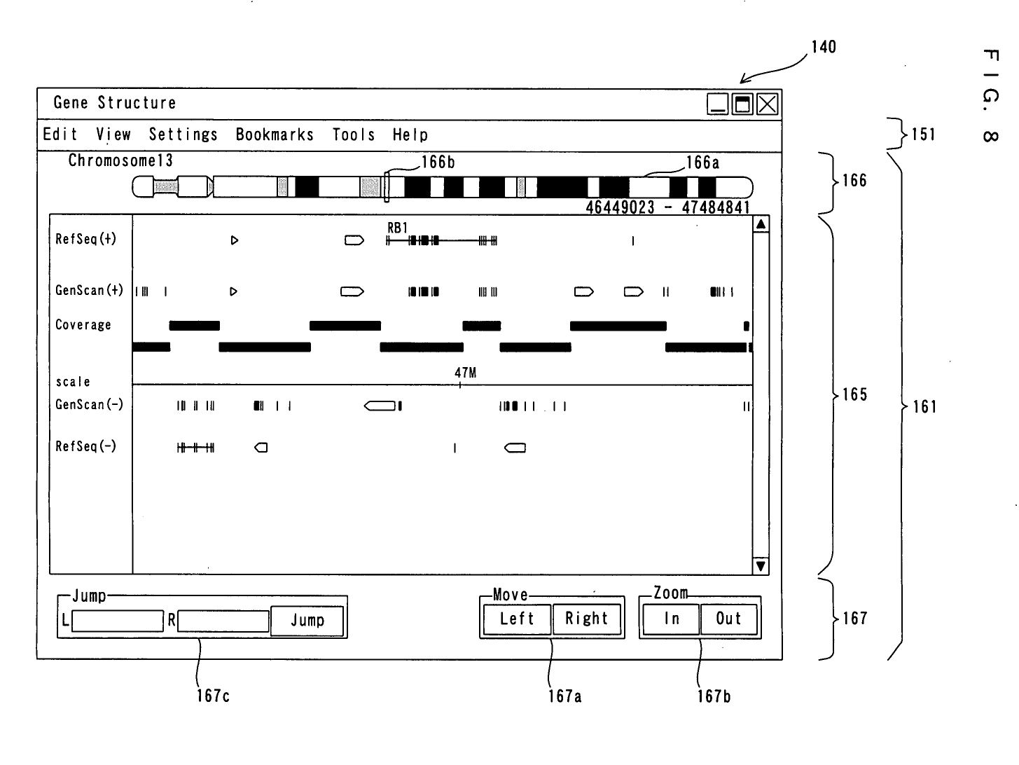

FIG. 8 shows an exemplary map screen. Specifically, the map screen 140 shown in FIG. 8 is obtained when “RB1” (tumor suppressor gene) is selected as a desired search result data via the search result display screen 130, and “UCSC Golden Path” is selected as a desired layout script and single screen framing is selected as a desired screen framing via the search condition input screen 120 shown in FIG. 7. The map screen 140 is divided into a menu zone 151 and a canvas zone 161. The menu zone 151 is a zone where the menus for controlling the map screen 140 are presented. The canvas zone 161 is a zone for presenting genome information, and is divided into three fields, i.e., first, second and third fields 165, 166 and 167, respectively. The first field 165 is located at the center of the canvas zone 161, while the second field 166 and third field 167 are located at the top and bottom of the canvas zone 161, respectively.

On the first field 165, an enlarged map is displayed. The enlarged map graphically presents the extension in genome in which each element exists such that the element corresponding to the search result data selected on the search result display screen comes roughly at the center of the enlarged map, for each kind of map screen data. In FIG. 8, the gene “RB1” is presented at the center of the enlarged map enlarged by a specified magnification. In the portion labeled “scale” on the enlarged map, the scale of position with respect to the chromosome is indicated, that is, the abscissa of the enlarged map indicates the positions with respect to the chromosome. At the left end of the enlarged map, the titles of map screen data are presented along a vertical direction. If any one of map screen data includes the strand, that map screen data is distinguished by the strand and the two map screen data in accordance with the strand are presented. In the particular example shown in FIG. 8, five titles of map screen data, that is, RefSeq (+), GenScan (+), Coverage, GenScan (−) and RefSeq (−) are presented.

On a lane (row) corresponding to the portion where the each title of map screen data is presented, the extension in genome in which each element included in the map screen data exists is graphically presented. Specifically, the extension in genome in which each element exists is graphically depicted as a band. However, when the map screen display control means 63c finds that the extension of the element on the enlarged map exceeds a specified size, and that the specified kind of map screen data includes an exon data, on the preparation of an enlarged map, the map screen display control means 63c graphically presents the extension in genome in which each exon of the element exists, instead of the extension in genome in which the element exists, in the display extension for the map screen data including the exon data. Specifically, the extension in genome in which each exon exists is graphically depicted as band, and the bands representing these exons are connected to each other by a straight line. In the particular example shown in FIG. 8, the gene “RB1” in RefSeq(+) are graphically presented in form of a exon structure. As seen from above, since it is possible according to this embodiment to graphically present each element included in each map screen data for each kind of map screen data, it is easy to compare the contents of those map screen data.

The second field 166 includes an overall map 166a indicating, in form of a band structure, the extension in genome corresponding to the mass of map screen data transmitted by the server 10, and a rubber band (indicating means) 166b for indicating, on the overall map 166a, the extension in genome presented on an enlarged map. In the particular example shown in FIG. 8, since a specified kind of map screen data related with chromosome No. 13 of human genome have been transmitted by the server 10, the band structural diagram characteristic with chromosome No. 13 of human genome is presented as the overall map 166a. On the upper left corner of the second field 166, the name of overall map 166a is indicated, while on the lower right corner of the second field 166, the extension in genome presented in the enlarge map is indicated by numbers (the start and end positions of the base). In the particular example shown in FIG. 8, “Chromosome 13” is given as the name of overall map 166a, while the extension in genome presented in the enlarged map is represented by “46449023-47484841.”

When the user clicks a point on the overall map 166a with mouse, the rubber band 166b moves to the point selected by clicking, and the extension in genome presented on the enlarged map is moved to the extension indicated by the moved rubber band 166b. Specifically, when a point on the overall map 166a is clicked by means of mouse, the map screen display control means 63c moves the rubber band 166b to the point. Then, the map screen display control means 63c prepares a renewed enlarged map presenting the extension indicated by the moved rubber band 166b based on the mass of map screen data stored in the memory means 64, displays the renewed enlarged map on the first field 165.

As described above, it is possible to alter the extension in genome presented on the enlarged map by clicking a desired point on the overall map 166a. Alteration of the extension in genome presented on the enlarged map may be achieved by an alternative method. For example, if a user drags the rubber band 166b on an overall map 166a with mouse to drop it on a desired point, the map screen display control means 63c may prepare an enlarged map about the extension indicated by the dropped rubber band 166b, and display the enlarged map on the first field 165.

In the third field 167, there are three kinds of operation means available for altering the extension in genome presented on the enlarged map. The operation means consists of a moving operation means 167a, expanding/contracting operation means 167b and jumping operation means 167c.

The moving operation means 167a is for moving the extension in genome presented on the enlarged map. As shown in FIG. 8, the moving operation means 167a consists of two buttons, “Left” and “Right.” When the “Left” button is turned on, the map screen display control means 63c prepares, based on the mass of map screen data stored in the memory means 64, an enlarged map where the extension in genome moves leftward at a constant speed as long as the “Left” button is activated, and displays the enlarged map on the first field 165. On the other hand, when the “Right” button is turned on, the map screen display control means 63c prepares, based on the mass of map screen data stored in the memory means 64, an enlarged map where the extension in genome moves rightward at a constant speed as long as the “Right” button is activated, and displays the enlarged map on the first field 165.

The expanding/contracting operation means 167b is for expanding/contracting the extension in genome presented on the enlarged map. As shown in FIG. 8, the expanding/contracting operation means 167b consists of two buttons, “In” and “Out.” When the “In” button is turned on, the map screen display control means 63c prepares, based on the mass of map screen data stored in the memory means 64, an enlarged map where the extension in genome expands at a constant speed as long as the “In” button is activated, and displays the enlarged map on the first field 165. On the other hand, when the “Out” button is turned on, the map screen display control means 63c prepares, based on the mass of map screen data stored in the memory means 64, an enlarged map where the extension in genome contracts at a constant speed as long as the “Out” button is activated, and displays the enlarged map on the first field 165.

The jumping operation means 167c is for specifying a desired extension in genome by numbers to display an enlarged map in the first field 165 where the extension in genome as specified is indicated. The jumping operation means 167c consists of two position-input boxes via which the user can feed the positions of the right and left ends of the extension in genome, and “Jump” button. When appropriate numbers are fed into the two position-input boxes and “Jump” button is turned on, the map screen display control means 63c prepares, based on the mass of map screen data stored in the memory means 64, an enlarged map presenting the extension in genome indicated by the numbers fed into the two position-input boxes, and displays the enlarged map on the first field 165.

When the extension in genome presented on the enlarged map is altered via the operation means 167a, 167b, 167c, the position/size of the rubber band 166b in the second field 166 and the numbers defining the extension in genome presented on the enlarged map also change in association with the alteration.

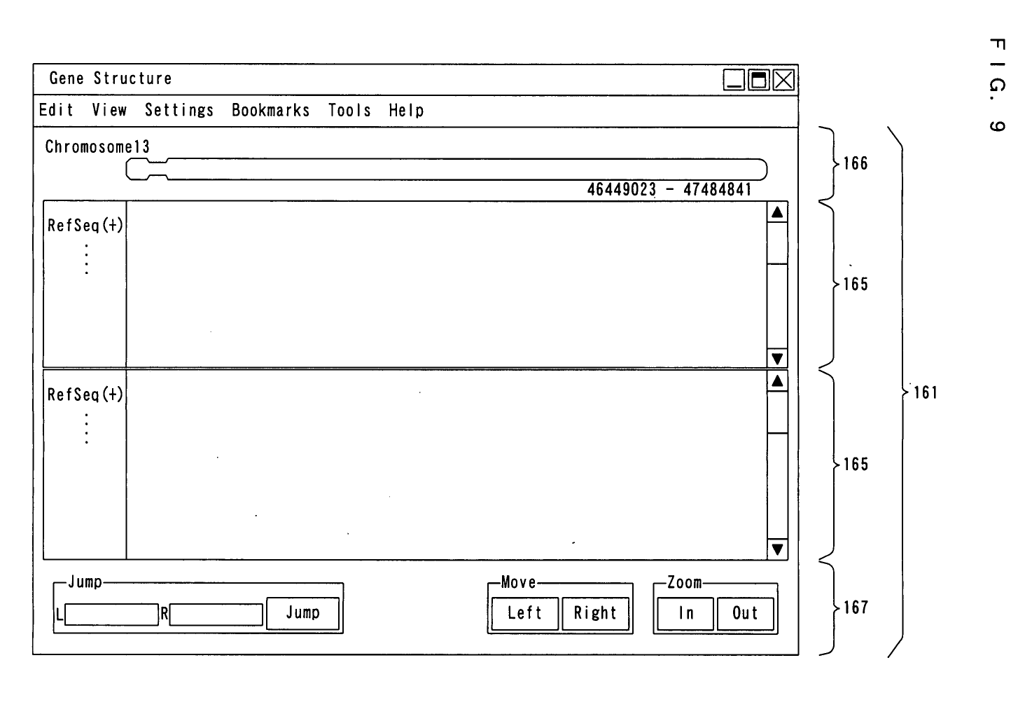

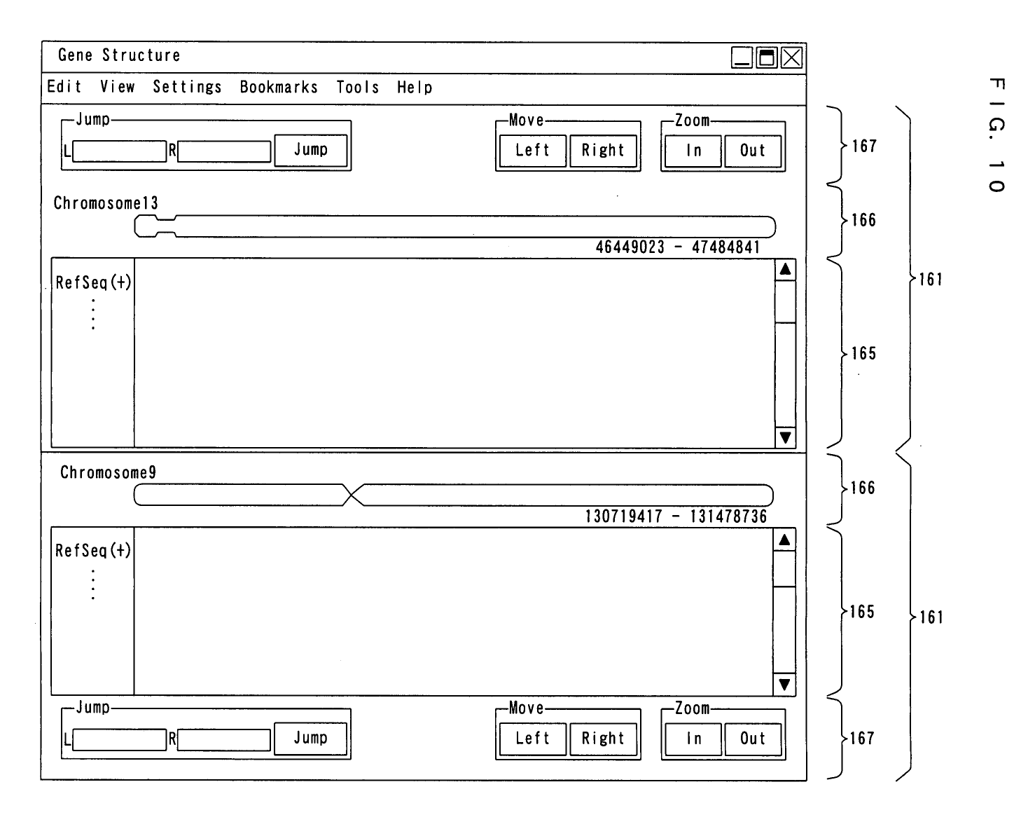

The map screen shown in FIG. 8 is based on single screen framing. According to this embodiment, synchronous screen framing and non-synchronous screen framing are available in addition to single screen framing. FIG. 9 shows an exemplary map screen presented as a synchronous screen framing. FIG. 10 shows an exemplary map screen presented as a non-synchronous screen framing. In FIGS. 9 and 10, parts of the overall map and enlarged map are omitted. As shown in FIG. 8, in single screen framing, the map screen includes a single canvass zone 161 in which there are one first field 165, one second field 166 and one third field 167. In synchronous screen framing, the map screen includes a single canvas zone 161 in which there are two first fields 165, 165 in addition to a single second field 166 and third field 167 as shown in FIG. 9. In the two first fields 165, 165 of the map screen, two enlarged maps with different magnifications prepared based on the same mass of map screen data, are displayed. In this map screen based on synchronous screen framing, only a single overall map and single set of operation means are available. When one of the operation means is operated in this map screen based on synchronous screen framing, the contents presented in the two first fields 165, 165 change simultaneously as instructed by the operation means. In non-synchronous screen framing, the map screen is as shown in FIG. 10: there are two canvas zones 161, 161 each of which includes one first field 165, one second field 166 and one third field 167. In short, in non-synchronous screen framing, the map screen consists of the two screens of the single screen framing. In the map screen based on non-synchronous screen framing, two canvas zones 161, 161 are independent of each other. Accordingly, if an operation means in one of the canvas zone 161 is operated, only the content presented in the first field 165 of the canvas zone 161 corresponding to the operation means alters as instructed by the operation means. Using a map screen based on such non-synchronous screen framing, it is possible to put side by side for comparison two or more kinds of genome information obtained from different organisms on the same screen.

Selection of the screen framing of map screen may be achieved as described above by means of the screen framing selection portion 123 in the search condition input screen 120. When synchronous screen framing is selected, it is necessary to determine the magnification of an enlarged map in the lower first field relative to an enlarged map in the upper first field. The relative magnification may be determined, for example, by selecting any one from x2, x5 or x10 options as shown in FIG. 7. When non-synchronous screen framing is selected, it is necessary to determine which one out of the upper and lower canvas zones is used for display of the current content. When the user wants to display the current content in the upper canvas zone, he/she may achieve it by selecting the “Upper” as shown in FIG. 7. Alternatively, when the user wants to display the current content in the lower canvas zone, he/she may achieve it by selecting the “Lower”.

The menu zone 151 of map screen 140 includes six menus, i.e., “Edit”, “View”, “Settings”, “Bookmarks”, “Tools” and “Help.” The “Edit” menu is for editing and reprocessing the element data, and includes three submenus, i.e., “Add annotation”, “Add element” and “Element style.” The “Add annotation” submenu is for attaching annotation to an element selected, and registering information of the annotation into the data set storage means 21. The annotation is an explanatory note attached to an element. The “Add element” submenu is for registering a new element into the data set storage means 21. The “Element style” submenu is for modifying the display attribute (color or shape) of an element on an enlarged map. The “View” menu is for visibly presenting sequence information and annotation information, and includes three submenus, i.e., “Nucleotide sequence”, “Amino acid sequence” and “Annotation.” The “Nucleotide sequence” submenu is for visibly displaying the base sequence of an element selected. The “Amino acid sequence” submenu is for visibly displaying the amino acid sequence of an element selected, provided that the amino acid sequence of the element is stored in the data set storage means 21. The “Annotation” submenu is for enabling the user to review annotation information attached to an element selected.

The “Settings” menu is involved in the control of display, etc., and includes two submenus “Lane” and “Filter.” The “Lane” submenu is for determining whether a lane selected is displayed or not on the enlarged map, or whether two lanes selected are interchanged or not on the enlarged map. The “Filter” submenu is for controlling the display of an enlarged map depending on the characteristic value of each element. For example, if certain kind of map screen data is selected from the multiple kinds of map screen data, and the range of characteristic values is notified, the map screen display control means 63c prepares, based on the characteristic value data included in a mass of map screen data stored in the memory means 64, en enlarged map which displays only elements whose characteristic values are within the notified range, on the selected kind of map screen data, and presents the enlarged map in the first field 165. As seen from above, using the “Filter” submenu, the user can narrow a range of the display objects. The “Bookmarks” menu is for registering a favorite display screen or jumping to a registered screen, and includes a submenu of “Bookmark.” The “Tools” menu includes a submenu of “BLAST” which is provided for performing a homology determination. The “Help” menu includes a submenu of “About This Application” which is provided for explaining the function of the system and its use.

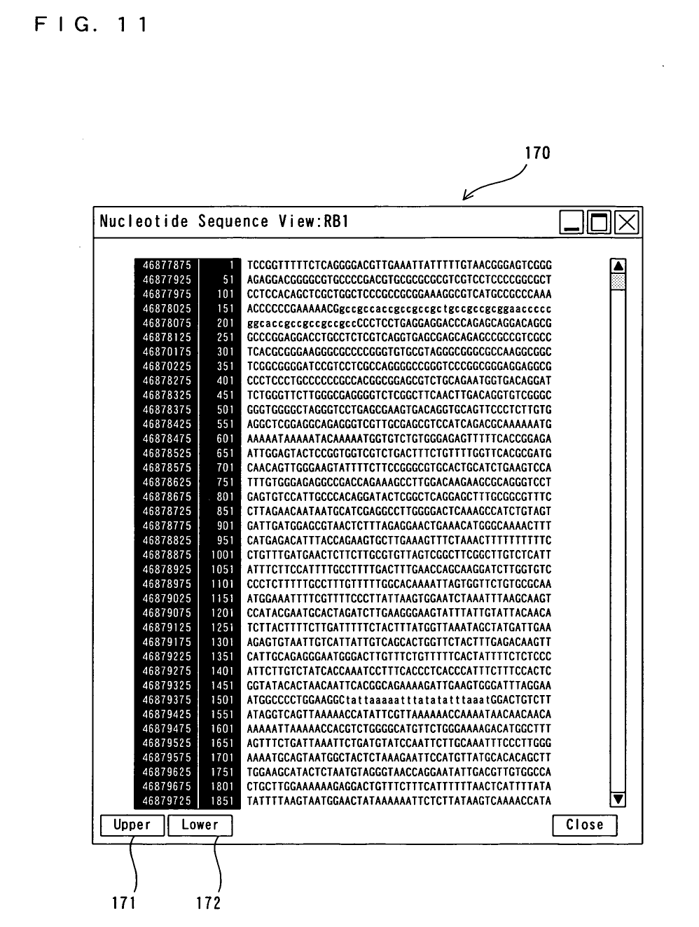

Here, description will be given in particular about the display screen of the base sequence that will be displayed when the user selects submenu “Nucleotide sequence”. FIG. 11 shows an exemplary display screen for indicating a base sequence. To see the base sequence of an element of interest in the display screen 170 of the base sequence, the user selects the element on an enlarged map. Then, the user clicks “View” menu to open a submenu including “Nucleotide sequence”. When the user selects “Nucleotide sequence”, the map screen display control means 63c prepares, based on base sequence data contained in a mass of map screen data stored in the memory means 64, a display screen 170 offering the base sequence of the selected element, and presents the base sequence display screen 170 in a screen distinct from the map screen. The base sequence display screen 170 presents the base sequence of the selected element in such a way that each row includes fifty bases. At the left end of each row, there are arranged two numbers: one far from the leading base in the row represents the position of the leading base in the chromosome, while the other close to the leading base in the row represents the position of the leading base in the element. If the element includes an exon(s), and the exon data is included in the mass of map screen data stored in the memory means 64, base sequences belonging to the exon will be presented in a different color from a color representing the sequences outside the exon so that the former can be distinguished from the latter. On the lower left corner of the base sequence display screen 170, there are two buttons, “Upper” 171 and “Lower” 172. Each time the “Upper” button 171 is activated, five hundred bases that are disposed at the upper side of the element will be displayed additionally. On the other hand, each time the “Lower” button 172 is activated, five hundred bases that are disposed at the lower side of the element will be displayed additionally.

The communication control means 63d is for transmitting, when the user feeds his/her user ID and password via the authentication screen 110 or when the user feeds search condition data via the search condition input screen 120, those data to the server 10. Furthermore, when one from the search result data presented on a search result display screen 130 is selected, the communication control means 63d transmits to the server 10 the chromosome number included in the selected search result data, the title of the layout script fed via the map screen data selection portion 121 and the content of the screen framing selected via the screen framing selection portion 123, together with a request for map screen related to the selected search result data.

According to the embodiment, the server 10 stores a display program in the data set storage means 21. The display program is for instructing a terminal 60 to execute functions assigned to the authentication screen display control means 63a, search screen display control means 63b, map screen display control means 63c, and communication control means 63d. The communication control section 45 of the sever 10, each time a terminal 60 makes a first access to the server 10, transmits the display program to the terminal 60. The terminal 60, on receipt of the display program from the server 10, loads it on its system. The terminal 60 has, for example, Java (Trade Mark or ™) Plug-in installed in a Web browser, and executes the display program on Java™ Plug-in. Thus, by way of the display program, functions are conferred on the terminal 60 which are executed by the authentication screen display control means 63a, search screen display control means 63b, map screen display control means 63c, and communication control means 63d. If the Web browser is closed, the program is usually cache-memorized in Java™ Plug-in, and thus it is not necessary for the terminal 60 to download the display program even when the terminal 60 makes a renewed access to the sever 10. Alternatively, it is possible to set Java™ Plug-in such that the loaded display program is not cache-memorized in Java™ Plug-in when the Web browser is closed. In this case, when the terminal 60 makes a renewed access to the server 10, it must download the display program anew from the server. Java™ Plug-in is one of the well-known programs provided by Sun Microsystems.

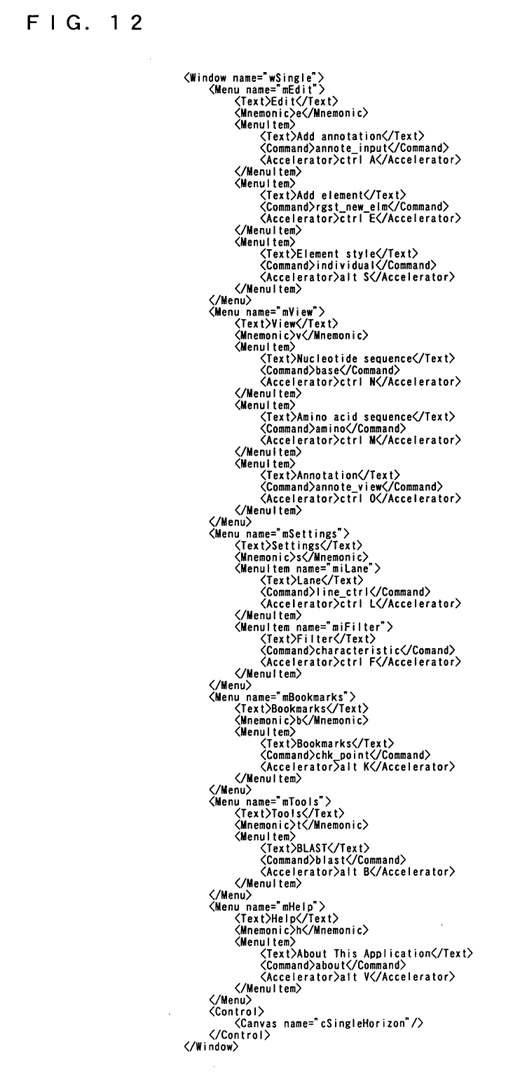

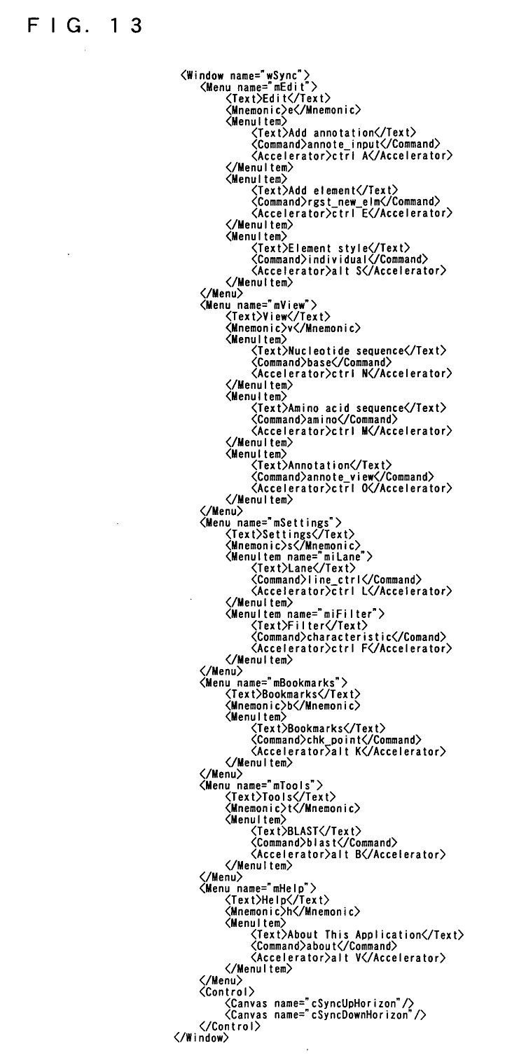

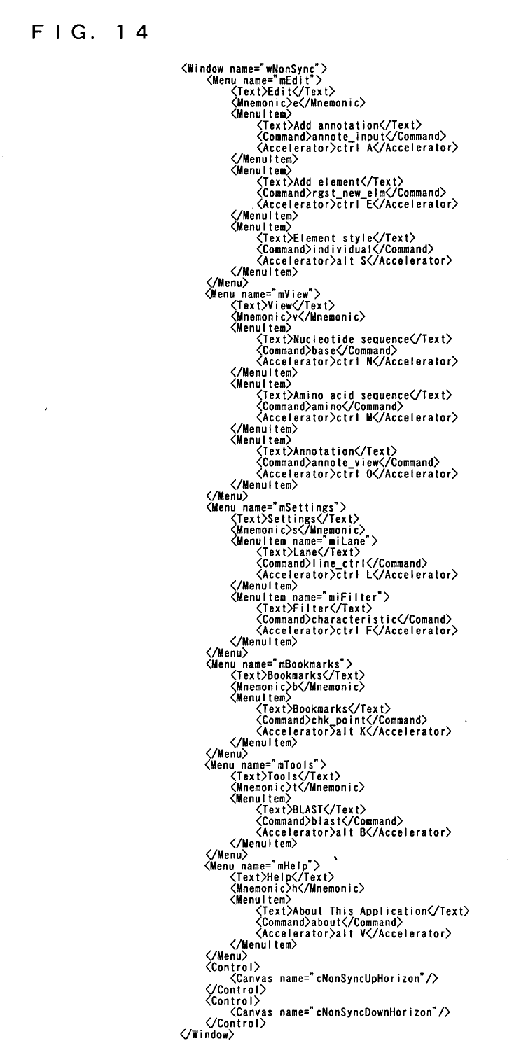

Next, the details of the screen framing script will be described below. FIG. 12 shows an exemplary screen framing script for a single screen framing. FIG. 13 shows an exemplary screen framing script for a synchronous screen framing. FIG. 14 shows an exemplary screen framing script for a non-synchronous screen framing.

The screen framing script determines the screen framing and menu arrangement of a map screen using tags. According to the description of a screen framing script, determined are which one is selected out of single screen framing, synchronous screen framing and non-synchronous screen framing as the screen framing of a map screen, which title is selected as a title of each menu presented in the menu zone 151 of a map screen, and which title is selected as a title of submenu presented on clicking each menu, etc. The tags utilized in a screen framing script include, for example, Window tag, Menu tag, MenuItem tag, Text tag, Mnemonic tag, Accelerator tag, Command tag, Control tag, Canvas tag, etc. The Window tag is the highest root tag. The Menu tag indicates a menu displayed in the menu zone 151. The MenuItem tag indicates an additional submenu included in a menu. The Text tag indicates the title of a menu or a submenu. The Mnemonic tag indicates a keyboard mnemonic for a menu. The Accelerator tag indicates a keyboard accelerator for a submenu. The Command tag indicates which command should be run when a certain submenu is clicked. The Control tag indicates an additional operation means. The Canvas tag indicates an additional canvas zone, and is provided at the lower layer of the Control tag. According to the notifications given in Control and Canvas tags, single screen framing, synchronous screen framing or non-synchronous screen framing is selected.

The screen framing scripts shown in FIGS. 12, 13 and 14 include six Menu tags at the lower layer of the Window tag, and those Menu tags are the same among the three screen framing scripts except for the number of Canvas tags provided at the last lower layer of the Window tag and contents attached to them. During the determination of the screen framing of a map screen using each of those screen framing scripts, six menus, that is, “Edit”, “View”, “Settings”, “Bookmarks”, “Tools” and “Help” are available on the menu zone 151 of the map screen. For each of the three screen framing scripts, for example, three MenuItem tags are provided at the lower layer of the Menu tag indicating “Edit”. Thus, in the map screen, the “Edit” menu includes the three submenus, i.e., “Add annotation”, “Add element” and “Element style” according to the descriptions given in the MenuItem tags. In the same manner, for each of other menus, the menu includes the submenus according to the descriptions given in the MenuItem tags that are provided at the lower layer of the Menu tag.

According to the screen framing script shown in FIG. 12, only one Control tag is provided at the lower layer of the Window tag and only one Canvas tag is provided at the lower layer of the Control tag. This requires single set of operation means, and the display extension in single canvas zone is altered by means of the set of operation means. Thus, according to the screen framing script shown in FIG. 12, single screen framing is selected for screen framing of map screen. On the other hand, according to the screen framing script shown in FIG. 13, only one Control tag is provided at the lower layer of the Window tag and two Canvas tags are provided at the lower layer of the Control tag. This requires single set of operation means, and the display extensions in two canvas zones are altered by means of the set of operation means. Thus, according to the screen framing script shown in FIG. 13, synchronous screen framing is selected for screen framing of map screen. Further, according to the screen framing script shown in FIG. 14, two Control tags are provided at the lower layer of the Window tag and only one Canvas tag is provided at the lower layer of the respective Control tags. This requires two sets of operation means, and the display extension in the canvas zone corresponding to each set of operation means is altered by means of the set of operation means. Therefore, according to the screen framing script shown in FIG. 14, non-synchronous screen framing is selected for screen framing of map screen.

The screen framing scripts as described above are stored in the setting data storage portion 27 of the data set storage means 21. The data management means 41 of the server 10, on receipt of the content selected via the screen framing selection portion 123 together with a request for map screen from a terminal 60, obtains the screen framing script corresponding to the selected content from the setting data storage portion 27 of the data set storage means 21, and transmits the screen framing script to the terminal 60. Then, the map screen display control means 63c of the terminal 60 determines, on receipt of the predetermined screen framing script together with a mass of map screen data necessary for the preparation of map screen from the server 10, the screen framing of the map screen based on the received screen framing script.

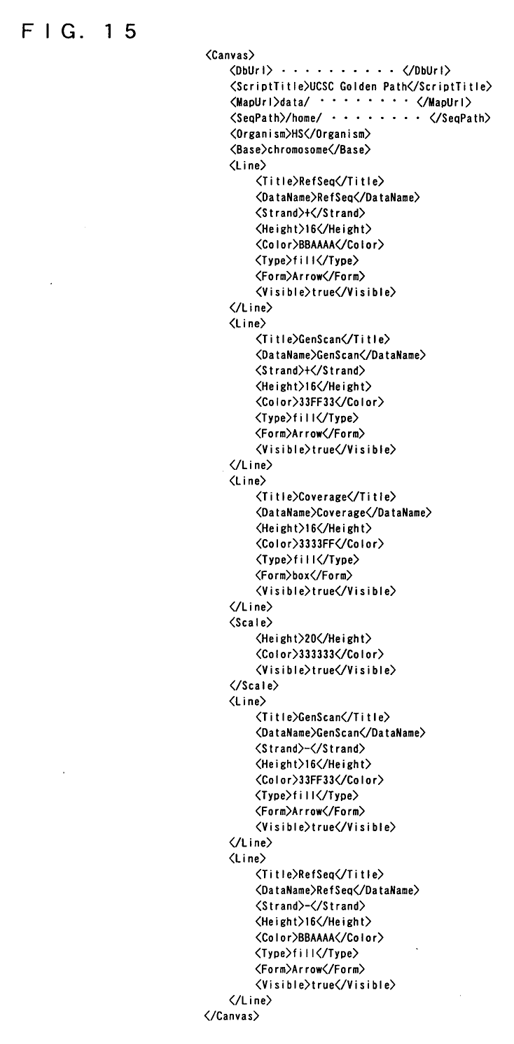

Next, the details of a layout script will be described below. FIG. 15 shows an exemplary layout script.

The layout script determines the layout of an enlarged map using tags. According to the description of a layout script, determined are which kinds of map screen data should be displayed on the enlarged map, in what order those kinds of map screen data should be arranged, in what size the display extension concerning each map screen data is pictured, and with what color the figures representing elements are pictured. The tags utilized in a layout script include, for example, Canvas tag, DbUrl tag, ScriptTitle tag, MapUrl tag, SeqPath tag, Organism tag, Base tag, Line tag, Scale tag, Title tag, DataName tag, Strand tag, Height tag, Color tag, Type tag, Form tag, Visible tag, etc. The Canvas tag is the highest-layer tag. The DbUrl tag indicates the URL for connection with the data set storage means 21. The ScriptTitle tag indicates a title presented in a combo box of the map screen data selection portion 121 in the search condition input screen 120. The MapUrl tag indicates the URL of the site where the data required for the display of an overall map presented in the second field 166 of map screen is stored. The SeqPath tag indicates the path to a directory where the map screen data necessary for the preparation of map screen is stored. Once this directory path is described in a program, it is not easy to change the path. Therefore, according to this embodiment, the path to a directory where the map screen data is stored is described in a layout script.

The Organism tag indicates the organism that the map screen data concerns. In this particular embodiment, the Organism tag indicates “HS”. “HS” means that the data concerns human genome. The Base tag indicates the basic unit of the map screen data necessary for the preparation of map screen. In this particular example, the Base tag indicates “chromosome”. This means that a mass of map screen data dealing with a chromosome as a basic unit is necessary to prepare the map screen. Alternatively, the Base tag may include “contig”. This means that a mass of map screen data dealing with a contig as a basic unit is necessary to prepare the map screen.

The Line tag indicates the map screen data displayed on the enlarged map. According to the descriptions given at the lower layer of the Line tag, how to display the map screen data, etc., are determined. The Scale tag indicates a scale (scale of position) displayed on the enlarged map. It is possible to include plural Line and Scale tags in the Canvas tag. According to order of the arrangement of the plural Line and Scale tags included in the Canvas tag, the map screen data and scale corresponding to those are displayed on the enlarged map.

The Title tag indicates the title (name) of map screen data displayed on the left side of the enlarged map. The DataName tag indicates the name of logic used for extracting data displayed on a lane. The Strand tag indicates the strand of the map screen data to be displayed. The Strand tag indicates either “+”, or “−”. According to the embodiment, if there are two same map screen data whose strands are opposite to each other, they are presented on different lanes even if they have the same title. The Height tag indicates in a numerical value a height level at which the map screen data or the scale should be presented. The Color tag indicates the color with which the map screen data or the scale should by present. The indication occurs in a hexadecimal number. The Type tag indicates, when the extension in genome in which each element exists is graphically displayed, in what type the graphical display should be pictured. For example, “line” or “fill” may be indicated as the picture type. The “line” is the picture type in which the extension of the graphical display is enclosed with lines, while the “fill” is the picture type in which the extension of the graphical display is painted over. The Form tag indicates, when the extension in genome in which each element exists is graphically displayed, in what form the graphical display should be pictured. For example, “box” or “arrow” may be indicated as the picture form. The “box” is the picture form in which the extension of the graphical display is pictured with a band, while the “arrow” is the picture form in which the extension of the graphical display is pictured with a band and an arrow whose direction coincides with the strand. The Visible tag indicates whether a lane or scale should be displayed (“true”) or not displayed (“false”) on the enlarged map when the map screen is initially prepared.

The layout scripts as described above are stored in the setting data storage portion 27 of the data set storage means 21. The data management means 41 of the server 10, on receipt of the title of a layout script selected via the map screen data selection portion 121 together with a request for map screen from a terminal 60, obtains the layout script corresponding to the selected title of a layout script from the setting data storage portion 27 of the data set storage means 21, and transmits the layout script to the terminal 60. Then, the map screen display control means 63c of the terminal 60 determines, on receipt of the predetermined layout script together with a mass of map screen data necessary for the preparation of map screen from the server 10, the layout of the first field in the map screen based on the received layout script. In fact, the layout of the enlarged map shown in FIG. 8 is determined according to the layout script described in FIG. 15.

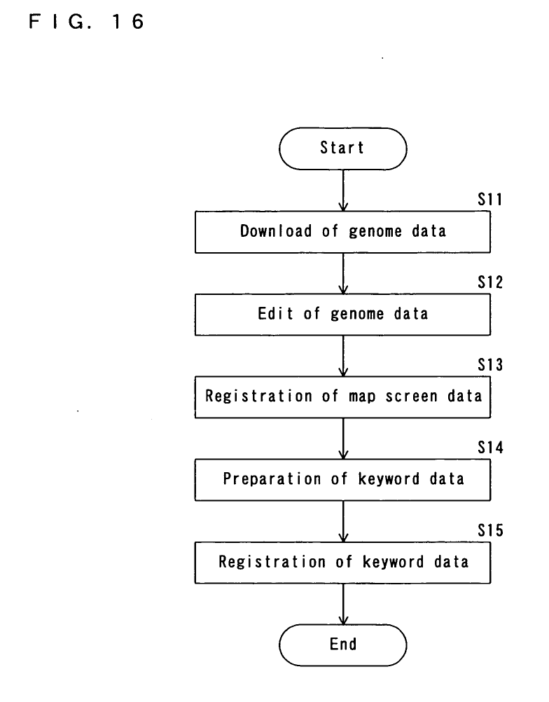

Next, the procedures for registering map screen data and keyword data into the data set storage means 21 of a genome information display system embodying the invention will be described. FIG. 16 is a flowchart for illustrating the procedures for registering map screen data and keyword data into the data set storage means 21 of a genome information display system representing an embodiment of the invention.

Registration of map screen data is performed by the map screen data registration/processing section 46, while registration of keyword data is performed by the keyword data registration/processing section 47. First, the map screen data registration/processing section 46 downloads genome data of a public database (S11). Needless to say, if map screen data is prepared based on the user's own genome data, the above procedure may be omitted. Next, the map screen data registration/processing section 46 edits the genome data thus downloaded according to a predetermined method (S12). Specifically, the map screen data registration/processing section 46 attaches an element ID to each of the elements included in the genome data, and extracts, for each element, its name, position, strand, comment, base sequence, characteristic values, etc., from the genome data. If the genome data includes the data of exons, the map screen data registration/processing section 46 attaches an exon number to each of the exons included in the element, and extracts, for each exon, its position data from the genome data. In some cases, on the extracted position data of elements or exons, the positions of elements or exons are determined with respect, for example, to a contig. In such a case, for example, the map screen data registration/processing section 46 converts the position with respect to a contig to the position with respect to a chromosome, and determines the positions of elements or exons in terms of the chromosome number and the start and end positions in the chromosome. Later, the map screen data registration/processing section 46 prepares, based on the data obtained as a result of editing as described above, the map screen data comprising the element data, exon data, base sequence data, site data, characteristic value data, etc., and registers those data into the map screen data storage portion 25 of the data set storage means 21 (S13).

Next, the keyword data registration/processing section 47 prepares keyword data based on the map screen data acquired by the map screen data registration/processing section 46 (S14). Specifically, the keyword data registration/processing section 47 extracts, for each element, its name and element ID from the element data included in the map screen data, attaches a keyword number to the extracted name of each element, and prepares keyword data using those extracted data and keyword numbers. Then, the keyword data registration/processing section 47 registers the keyword data of each element thus prepared into the keyword data storage portion 26 of the data set storage means 21 (S15).

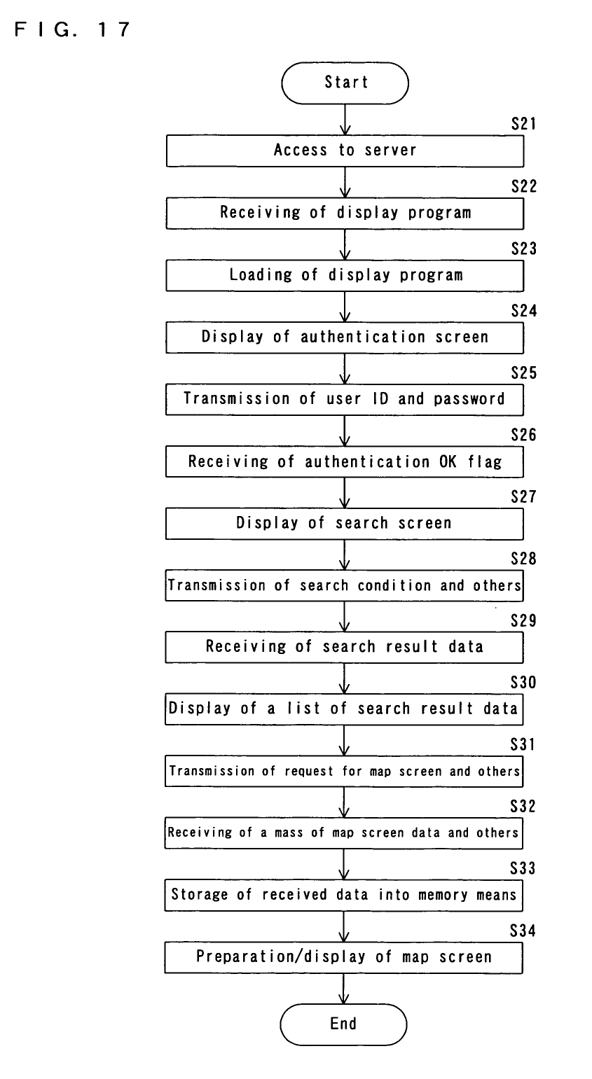

Next, the procedures for displaying a map screen on the display means 62 of a terminal 60 in a genome information display system embodying the invention will be described. FIG. 17 is a flowchart for illustrating the procedures for displaying a map screen on the display means 62 of the terminal 60.

First, the user runs a Web browser on his/her terminal 60. Then, the user enters, on the Web browser, the URL for the top page of the server 10 to gain access to the server 10 (S21). Each time receiving access from a terminal 60, the server 10 transmits a display program to the terminal 60. On receipt of the display program from the server 10 (S22), the terminal 60 loads the program (S23). As a result of this procedure, the program is incorporated in the terminal 60 so that its function becomes achievable on the terminal 60. Specifically, functions of displaying an authentication screen and search screen, and preparing and displaying a map screen are conferred to the terminal 60. If the terminal 60 has already a display program in cache, the steps S22 and S23 may be omitted, and the terminal 60 may proceed, on receipt of a response from the server 10 on the procedure for step S21, to the step S24.

Then, the authentication screen display control means 63a of the terminal 60 causes an authentication screen 110 to be displayed on the display means 62 (S24). The user enters his/her user ID and password on the authentication screen 110, and clicks “OK” button 113. The communication control means 63d of the terminal 60 transmits an authentication request together with the user ID and password to the server 10 (S25).

The server 10 carries out authentication procedure using the user ID and password transmitted by the terminal 60. Specifically, the server 10 checks whether the user ID and password currently transmitted by the terminal 60 are registered in the user management data storage portion 28 of the data set storage means 21. Finding that the user ID and password in question are registered, the server 10 authenticates the user, permits the access of the user, and transmits an authentication OK flag to the terminal 60. This makes it possible for the server 10 and the terminal 60 to communicate data with each other. On the contrary, if the server 10 determines that the user ID and password are not registered, the server 10 transmits an authentication FAIL flag to the terminal 60.

On receipt of an authentication OK flag from the server 10 (S26), the search screen display control means 63b of the terminal 60 causes a search condition input screen 120 and search result display screen 130 to be displayed on the display means 62 (S27). The user clicks the left-end button of the map screen data selection portion 121 in the search condition input screen 120 to open a list of the titles of layout scripts, and selects a desired layout script. Then, the user enters, in the search condition input portion 122, the name of an element to be displayed (search condition). The user selects desired screen framing for map screen in the screen framing selection portion 123. On completion of input feeding, the user clicks “Search” button 124. Then, the communication control means 63d transmits, to the server 10, a search request together with the title of a layout script selected via map screen data selection portion 121 and search condition fed via search condition input portion 122 (S28).

On receipt of a search request together with the title of a layout script and search condition from the terminal 60, the search means 31 of the server 10 starts search procedure. Specifically, the search means 31 of the server 10 acquires layout script whose title is the same as that of the layout script transmitted from the setting data storage portion 27 of the data set storage means 21. The search means 31 identifies, based on the content of the thus acquired layout script, several kinds (titles) of map screen data through which search should be made. Then, the search means 31 searches through keyword data for the data including the keyword that corresponds with the search condition. This search is performed on the keyword data including the names that are the same as the titles of map screen data previously identified by the search means 31. The search means 31 extracts the name of data and element ID included in each keyword data obtained as a result of the search, and acquires the element data related to the extracted name of data and element ID from the plural map screen data storage portions 25. The search result data consists of the thus obtained element data and the titles map screen data that include those element data. Specifically, the search result data includes, for each element selected according to the search condition, the name of data, element ID, name of element, chromosome number, start position, end position, strand, and comment. The communication control section 45 of the server 10 transmits the search result data to the terminal 60.

On receipt of the search result data from the server 10 (S29), the search screen display control means 63b of the terminal 60 causes a list of search result data to be displayed on a search result display screen 130 (S30). The user selects a desired search result data from the list of search result data. Then, the communication control means 63d transmits, to the server 10, a request for map screen related to the selected search result data together with the chromosome number included in the selected search result data, title of the layout script selected via the map screen data selection portion 121 and content of the screen framing selected via the screen framing selection portion 123 (S31).

On receipt of a request for map screen related to a search result data from terminal 60, the server 10 acquires the required data from the data set storage means 21. Specifically, the data management means 41 of the server 10 obtains first a screen framing script corresponding to the content of the screen framing transmitted together with the request for map screen, from the setting data storage portion 27. Then, the data management means 41 acquires a layout script whose title is the same as that of the layout script transmitted together with the request for map screen, from the setting data storage portion 27. The data management means 41 identifies the content described in the Base tag of the layout script. For illustration, let's assume “chromosome” is indicated by the Base tag. Then, the data management means 41 knows that preparation of a map screen requires a mass of map screen data dealing with a chromosome as a basic unit. Next, the data management means 41 identifies the titles of map screen data described in the “Line” tags of the layout script. The data management means 41 extracts, from the map screen data storage portions 25 in which the map screen data having the identified titles are stored, all of the element data having the chromosome number identical with the chromosome number transmitted by the terminal 60. The same means 41 also extracts, in addition to the element data, all of the exon data, base sequence data, site data and characteristic value data which include the element ID identical with the element ID included in the extracted element data. These extracted data constitute a mass of map screen data necessary for the preparation of map screen related to the selected search result data.

Then, the data management means 41 transmits the mass of map screen data necessary for the preparation of map screen related to the selected search result data together with the obtained screen framing script and layout script to the terminal 60. Certain kinds of map screen data may lack exon data, site data or characteristic value data. In such cases, naturally the data that don't exist in the kinds of map screen data are not transmitted. For preparing a map screen, it is necessary to have at least the element data. Thus, a mass of map screen data does not fail to include the element data.

On receipt of a mass of map screen data necessary for the preparation of map screen together with the screen framing script and layout script from the server 10 (S32), the terminal 60 stores those data into its memory means 64 (S33). The map screen display control means 63c of the terminal 60 then prepares a map screen based on the data, and causes the map screen to be displayed on the display means 62 (S34). Specifically, the map screen display control means 63c first determines whether the screen framing of map screen should be single screen framing, synchronous screen framing or non-synchronous screen framing based on the screen framing script transmitted by the server 10, and then determines the layout of enlarged map based on the layout script transmitted by the server 10. The map screen display control means 63c frames the map screen according to the screen framing determined as above, prepares an enlarged map such that the element corresponding to the search result data selected in the search result display screen 130 comes roughly at the center of the enlarged map, for each kind of map screen data, based on the mass of map screen data transmitted by the server 10 as well as on the layout selected as above, and causes the enlarged maps to be displayed on the first field of the map screen. In addition, the map screen display control means 63c refers to the description of the layout script, finds the URL of a site where the image data of an overall map is stored, and acquires the image data of the overall map from the site. The map screen display control means 63c causes an overall map corresponding to a chromosome number included in the search result data selected via the search result display screen 130 to be displayed on the second field of the map screen. The map screen display control means 63c further causes a rubber band 166b to indicate, on the overall map, the extension in genome presented in the first field, and causes numerical values representing the extension to appear at the lower right corner of the second field. Still further, the map screen display control means 63c causes a moving operation means 167a, expanding/contracting operation means 167b and jumping operation means 167c to be displayed on the third field of the map screen. Thus, a map screen as shown in FIG. 8 is presented on the screen of the terminal 60.

Next, operations achievable on the map screen 140 will be described. Operations achievable on the map screen 140 are divided into those achievable on the canvas zone 161 and those achievable on the menu zone 151. According to this embodiment, a mass of map screen data necessary for the preparation of map screen is transmitted in advance by the server 10 to the terminal 60 and stored in the memory means 64 of the terminal 60. Because of this, when the user conducts a desired operation on the map screen 140, it is not necessary for the terminal 60 to make a renewed access to the server 10 for further data communication. And, the terminal 60 can quickly carry out the processing corresponding to the user's operation, based on the mass of map screen data stored in the memory means 64.

Operations achievable on the canvas zone 161 include movement and expansion/contraction of the display extension of an enlarged map. These operations can be achieved by means of a moving operation means 167a, expanding/contracting operation means 167b and jumping operation means 167c provided in the third field. When the user turns on the “Left” button (or “Right” button) of the moving operation means 167a for example, the extension in genome displayed on the enlarged map moves leftward (or rightward) at a constant speed as long as the button is activated. Namely, the map screen display control means 63c prepares, based on the mass of map screen data stored in the memory means 64, an enlarged map where the extension in genome moves leftward (rightward) at a constant speed as long as the button is activated, and displays the enlarged map on the first field. When the user turns on the “In” button (or “Out” button) of the expanding/contracting operation means 167b, the extension in genome displayed on the enlarged map zooms in (or zoom out) as long as the button is activated. Namely, the map screen display control means 63c prepares, based on the mass of map screen data stored in the memory means 64, an enlarged map where the extension in genome zooms in (or zooms out) at a constant speed as long as the button is activated, and displays the enlarged map on the first field. When the user feeds appropriate numbers into the two input boxes of the jumping operation means 167c, and clicks the “Jump” button, the enlarged map presenting the extension in genome indicated by the fed numbers is instantly displayed. Namely, the map screen display control means 63c prepares, based on the mass of map screen data stored in the memory means 64, an enlarged map presenting the extension in genome indicated by the fed numbers, and displays the enlarged map on the first field.

It is also possible to move the display extension of an enlarged map using the rubber band 166b. When the user clicks a point on the overall map 166a with mouse, the rubber band 166b moves to the point selected by clicking, and the enlarged map presenting the extension indicated by the moved rubber band 166b is instantly displayed. Namely, when a point on the overall map 166a is clicked by means of mouse, the map screen display control means 63c moves the rubber band 166b to the point selected by clicking, prepares an enlarged map presenting the extension indicated by the moved rubber band 166b based on the mass of map screen data stored in the memory means 64, and displays the enlarged map on the first field.

The operation achievable on the canvas zone 161 further includes the display of link information. When the user places cursor of mouse on an element on an enlarged map and right-clicks mouse, the map screen display control means 63c acquires the site data related to the element from a mass of map screen data stored in the memory means 64, and causes the URL link information concerning the element to be displayed. If the user selects one of the URL link information, the communication control means 63d gains access to the site based on the URL link information, and thus the Web page of the site is presented on display. In this way, the user can easily get information about a given element.

Next, operations achievable on the menu zone 151 include the display of base sequence, display control based on characteristic value, etc. In order to have a display of base sequence, the user selects an element by clicking it on the map screen 140. Then, the user clicks the “View” menu to open a list of submenus, and clicks the “Nucleotide sequence” in the submenus. The map screen display control means 63c then fetches the base sequence data corresponding to the element thus selected from a mass of map screen data stored in the memory means 64, and displays a screen indicating a base sequence as shown in FIG. 11.

In order to control the display based on a characteristic value, the user clicks the “Setting” menu to open submenus, and clicks the “Filter” in the submenus. Then, the map screen display control means 63c displays a list of the titles of map screen data displayed at the lanes on the enlarged map. When the user selects a title on the list, the map screen display control means 63c presents a screen for setting the range of a characteristic value. When the user sets the range of a characteristic value on the screen, the map screen display control means 63c, based on the mass of map screen data stored in memory means 64, prepares an enlarged map presenting only the elements whose characteristic values fall within the set range in the selected map screen data, and displays the enlarged map on the first field. Control of the display based on a characteristic value cannot be applied to map screen data having no characteristic value data.

If the user clicks the closure button on the upper right corner of map screen 140, the map screen 140 will be closed. If the user selects “Close” of the file menu of Web browser, or clicks the closure button on the upper right corner of Web browser, the Web browser will be closed, and then, a communicative state between the terminal 60 and the server 10 dissolves. When a communicative state between the terminal 60 and the server 10 dissolves, all the data transmitted by the server 10 while the terminal 60 and the server 10 were in the communicative state are erased from the memory means 64. This is because the amount of data on genome is so enormous that it is necessary to avoid the wasteful use of a limited capacity of the memory means 64 of the terminal 60.

According to the genome information display system embodying the invention, on receipt of a request for map screen from a terminal, the server transmits a mass of map screen data necessary for the preparation of map screen to the terminal. Therefore, for example, when the user conducts operations such as movement, expansion/contraction of the display extension of an enlarged map on a map screen, the terminal, instead of making a renewed access to the server for further data communication, quickly prepares an enlarged map presenting the display extension according to the operation, based on the mass of map screen data stored in the memory means, and displays the enlarged map at a high speed matched with the user's operation. In addition, according to the embodiment, the server manages map screen data and keyword data and so on, and whenever the terminal makes a access to the server, the server transmits appropriate data to the terminal. This enables the efficient management of data and ensures the economical use of the resources of the terminal.

The present invention is not limited to the embodiment described above, and it will be possible to develop many modifications based on the embodiment without departing from the scope of the invention.

For example, the embodiment may be modified such that the user can freely set the content of a layout of the first field of the map screen in his/her own terminal, and the terminal transmits the setting data to the server so that the server can register the setting data as a layout script into its data set storage means. This allows the user to customize the layout of map screen and enables the user's easy handling of the layout of map screen. In this connection, the layout scripts prepared by individual users are preferably stored with classification by users. Through this arrangement, it is possible for a given user to exclusively use a layout script set by him/herself.

The above embodiment has been described on the premise that the display program for instructing a terminal to execute functions assigned to the authentication screen display control means, search screen display control means and map screen display control means is stored in the data set storage means of the server, and the server transmits, each time a terminal makes a first access to the server, the display program to the terminal. However, the display program may be, for example, a program for instructing a terminal to execute functions assigned only to the search screen display control means and map screen display control means. In this case, a program on authentication should be installed in advance in each terminal. This display program may be transmitted from the server to a terminal, after the server verifies the authentication of the user. Alternatively, the display program may be a program for instructing a terminal to execute function assigned only to the map screen display control means. In this case, a program on authentication and search should be installed in advance in each terminal. This display program may be transmitted from the server to a terminal, when the server receives a request for map screen from the terminal, together with a mass of map screen data necessary for the preparation of map screen. Alternatively, transmission of a display program from the server to the terminal may be omitted, and a program enabling the execution of functions assigned to the authentication screen display control means, search screen display control means, and map screen display control means may be installed in advance in each terminal.

The above embodiment has been described on the premise that the search condition input screen includes the screen framing selection portion for selecting a desired screen framing for map screen from among single screen framing, synchronous screen framing and non-synchronous screen framing. However, the screen framing selection portion may be for selecting a desired screen framing for map screen from among single screen framing and synchronous screen framing. Alternatively, the screen framing selection portion may be for selecting a desired screen framing for map screen from among single screen framing and non-synchronous screen framing.

The above embodiment has been described on the premise that the content on the screen framing of map screen are separated from the program for the preparation of map screen, and stored as screen framing script in the data set storage means. However, the content on the screen framing may be specified in the program for the preparation of map screen. The above embodiment has been described on the premise that the content on the layout of an enlarged map are separated from the program for the preparation of map screen, and stored as layout script in the data set storage means. However, the content on the layout of an enlarged map may be specified in the program for the preparation of map screen.

The above embodiment has been described on the premise that a mass of map screen data principally dealing with a chromosome as a basic unit is transmitted from the server to a terminal. However, a mass of map screen data dealing with a contig as a basic unit may be transmitted from the server to a terminal. What is the basic of a mass of map screen data can be indicated by the Base tag of a layout script as described above. Let's assume, for illustration, that the Base tag of a layout script indicates “contig”. Then, in the search result display screen 130 shown in FIG. 7, an item “contig” is substituted for the item “Chrom” and the number of contig to which the element belongs is presented in the item “contig”. Also, a mass of map screen data dealing with the entire genome as a basic unit may be transmitted from the server to a terminal.

And it is a matter of course that an object of the present invention can be attained also by supplying a program code (including its execution form) of a software program for realizing functions of an apparatus according to the above-mentioned embodiment to an apparatus by means of a storage medium recording the whole or a part of the program code on it, making a computer (or CPU, MPU) of the apparatus read the program code stored in the storage medium, and performing the whole or a part of its operation. In this case the program code itself read out from the storage medium comes to realize the functions of this embodiment, and the storage medium storing the program code in it results in composing the present invention.

As a storage medium for supplying a program code, it is possible to use a ROM, floppy (™) disk, hard disk, optical disk, magneto-optic disk, CD-ROM, CD-R, DVD-ROM, magnetic tape, nonvolatile memory card and the like. Further, a program code may be supplied by being downloaded through a communication circuit, and a program code can be supplied and executed by utilizing a technique of JAVA™ and the like.

And it is a matter of course that the present invention includes not only a case that the functions of this embodiment are realized by executing a program code read by a computer but also a case that an OS or the like running on a computer performs a part or the whole of an actual process on the basis of instructions of the program code and thereby the functions of this embodiment are realized.

Furthermore, it is a matter of course that the present invention includes also a case that a program code read out from a storage medium is written into a memory provided on a function extending board inserted into a computer or a function extending unit connected to a computer and thereafter a CPU or the like provided in the function extending board or the function extending unit performs a part or the whole of an actual process on the basis of instructions of the program code and the functions of this embodiment are realized by this process.

Additionally, the present invention may be a program product including a program for making a computer realize functions of an apparatus according to the above-mentioned embodiment. Here, a program product includes not only a computer program but also a storage medium or a computer recording the program in it.

As described above, according to a genome information display system of the invention, on receipt of a request for map screen from a terminal, the server transmits a mass of map screen data necessary for the preparation of map screen to the terminal. Therefore, for example, when the user conducts operations such as movement, expansion/contraction of the display extension of an enlarged map on a map screen, the terminal, instead of making a renewed access to the server for data communication, quickly prepares an enlarged map presenting the display extension according to the operation, based on the mass of map screen data stored in the memory means, and displays the enlarged map at a high speed matched with the user's operation. Accordingly, the present invention is particularly suitable as means for reviewing genome information for those who make research on genome.

1. A genome information display system comprising a server which manages plural kinds of map screen data each kind of map screen data including the position data of individual elements constituting a genome, and plural terminals which are connected to the server via a network such that a map screen for the graphical representation of each element is displayed on a display means of each terminal, wherein the server comprises:
data set storage means for storing the plural kinds of map screen data;
search means which, on receipt of search condition data from each terminal in the quest of data stored within the data set storage means, searches through the data stored within the data set storage means for the required data based on the search condition data; and
data management means which transmits the search result data obtained as a result of the search by the search means to the terminal, and on receipt of a request for map screen related to a search result data from the terminal, fetches a mass of map screen data necessary for preparing the requested map screen related to the search result data from the data set storage means to transmit that mass of map screen data to the terminal,
and wherein each terminal comprises:
first display control means which causes a search condition input screen through which the user can enter search condition data in the quest of data within the data set storage means, and a search result display screen for displaying search result data transmitted from the server to be displayed on the display means;
communication control means which transmits search condition data fed via the search condition input screen to the server, and transmits, when the user selects one of the search result data presented on the search result display screen, a request for map screen related to the selected search result data to the server;
memory means for temporarily storing a mass of map screen data necessary for the preparation of map screen transmitted by the server; and
second display control means which, on receipt of a mass of map screen data necessary for the preparation of map screen related to the search result data selected on the search result display screen from the server, prepares an enlarged map graphically presenting the extension in genome in which each element exists such that the element corresponding to the selected search result data comes roughly at the center of the enlarged map, for each kind of map screen data, based on the mass of map screen data transmitted by the server, and causes the enlarged map to be displayed on a first field of the map screen, further, prepares an overall map for presenting the extension in genome corresponding to the mass of map screen data transmitted by the server in form of a band structure, with an indicating means for indicating, on the overall map, the extension in genome presented on the enlarged map, and causes the overall map with the indicating means to be displayed on a second field of the map screen, and furthermore, when the user moves the indicating means on the overall map by operation of an input means, prepares the enlarged map presenting the extension indicated by the moved indicating means based on the mass of map screen data stored within the memory means, and causes the enlarged map to be displayed on the first field.

2. A genome information display system as described in claim 1 wherein a moving operation means for moving the extension in genome presented on the enlarged map is provided in a third field of the map screen, and when the operation of the moving operation means is conducted, the second display control means prepares the enlarged map where the extension in genome to be displayed is moved at a specified speed as long as the operation is conducted, based on the mass of map screen data stored within the memory means, and causes the enlarged map to be displayed on the first field.

3. A genome information display system as described in claim 1 wherein an expanding/contracting operation means for expanding or contracting the extension in genome presented on the enlarged map is provided in a third field of the map screen, and when the operation of the expanding/contracting operation means is conducted, the second display control means prepares the enlarged map where the extension in genome to be displayed is expanded or contracted at a specified speed as long as the operation is conducted, based on the mass of map screen data stored within the memory means, and causes the enlarged map to be displayed on the first field.

4. A genome information display system as described in claim 1 wherein a jumping operation means for specifying the extension in genome by numeric value and displaying the enlarged map about the specified extension in genome on the first field is provided in a third field of the map screen, and when the operation of the jumping operation means is conducted, the second display control means prepares the enlarged map about the extension in genome specified by numeric value via the jumping operation means based on the mass of map screen data stored within the memory means, and causes the enlarged map to be displayed on the first field.

5. A genome information display system as described in claim 2 wherein either the search condition input screen or the search result display screen includes a screen framing selection means for selecting screen framing of the map screen, and the second display control means prepares the map screen of the screen framing selected by the screen framing selection means at the time of the display of the map screen.

6. A genome information display system as described in claim 5 wherein it is possible to select, by way of the screen framing selection means, single screen framing where the map screen consists of one first field, one second field and one third field.

7. A genome information display system as described in claim 5 wherein it is possible to select, by way of the screen framing selection means, synchronous screen framing where the map screen consists of two first fields, one second field and one third field, and the two enlarged maps with different magnifications prepared based on the same mass of map screen data are respectively displayed on each of the first fields.

8. A genome information display system as described in claim 5 wherein it is possible to select, by way of the screen framing selection means, non-synchronous screen framing where the map screen comprises the two screens of the single screen framing.

9. A genome information display system as described in claim 5 wherein the data set storage means stores plural screen framing scripts defining the content of the screen framing of the map screen, the data management means acquires, on receipt of the content selected by the screen framing selection means together with a request for map screen from each terminal, the screen framing script corresponding to the selected content from the data set storage means and transmits the screen framing script to the terminal, and the second display control means determines, on receipt of the predetermined screen framing script together with the mass of map screen data necessary for the preparation of map screen transmitted from the server, the screen framing of the map screen based on the received screen framing script.

10. A genome information display system as described in claim 1 wherein the data set storage means stores plural layout scripts defining the content of the layout of the enlarged map, the search condition input screen includes a layout script selection means for selecting a layout script, and the search means acquires, on receipt of the search condition data including the content selected by the layout script selection means from each terminal, the layout script corresponding to the selected content from the data set storage means, and identifies the kind of map screen data to be searched based on the content of the acquired layout script.

11. A genome information display system as described in claim 10 wherein the data management means acquires, on receipt of the content selected by the layout script selection means together with a request for map screen from each terminal, the layout script corresponding to the selected content from the data set storage means and transmits the layout script to the terminal, and the second display control means determines, on receipt of the predetermined layout script together with the mass of map screen data necessary for the preparation of map screen transmitted from the server, the layout of the enlarged map based on the received layout script.

12. A genome information display system as described in claim 1 wherein if the extension of a element on the enlarged map exceeds a specified size, and the specific kind of map screen data includes position data of each exon, on the preparation of enlarged map, the second display control means graphically presents the extension in genome in which each exon of the element exists, instead of the extension in genome in which the element exists, in the display extension of the element for the map screen data including the position data of each exon.

13. A genome information display system as described in claim 1 wherein the data set storage means stores a display program which enables each terminal to execute functions assigned to the first and second display control means, and the data management means transmits, on receipt of a first access from each terminal, the display program to the terminal, and each terminal loads the display program transmitted from the server.

14. A computer-readable recording medium on which is recorded a program for enabling a computer to execute the functions of a genome information display system as described in claim 1.

15. A program for enabling a computer to execute the functions of a genome information display system as described in claim 1.
